(19) United States
(12) Patent Application Publication
(10) Pub. No.: US 2026/0367088 A1
(43) Pub. Date: Nov. 19, 2026

(54) REACTIVE PROBES, PULL-DOWN, AND PURIFICATION METHODS FOR TAGGING BIOMOLECULES

(71) Applicant: SYNCELL (TAIWAN) INC., Taipei City (TW)

(72) Inventors: Jung-Chi LIAO, Taipei City (TW); Hsiao-Jen CHANG, Taipei City (TW)

(21) Appl. No.: 19/124,769

(22) PCT Filed: Oct. 27, 2023
(86) PCT No.: PCT/US2023/078017

§ 371 Date: Apr. 25, 2025

Related U.S. Application Data
(60) Provisional application No. 63/381,188, filed on Oct. 27, 2022.

(51) Int. Cl. G01N 33/58; G01N 30/02; G01N 30/72; G01N 30/88; G01N 33/68

(52) U.S. Cl. CPC G01N 33/58; G01N 30/7233; G01N 30/88; G01N 33/6848; G01N 2030/027; G01N 2030/8831

(57) ABSTRACT

Provided herein are compositions, kits, and methods useful for photolabeling and enrichment of biomolecules of interest. The probes of the composition may include reactive probes. The reactive probes may be used for labeling molecules and in pull-down and purification methods for subsequent analyses.

### CROSS REFERENCE OF RELATED APPLICATION

This application claims priority to U.S. Provisional Patent Application No. 63/381,188, filed on Oct. 27, 2022, titled “REACTIVE PROBES AND PULL-DOWN METHODS FOR TAGGING BIOMOLECULES,” which is herein incorporated by reference in its entirety.

### TECHNICAL FIELD

Described herein are reactive probes, pull-down, and purification methods that may be useful for chemical reaction labeling, and kits which include the reactive probes. Specifically described are photoreactive probes useful for labeling subsets of biomolecules. The probes may be particularly useful for analyzing biological samples, such as identifying proximal biomolecules in cell or tissue samples, and the method may be helpful for improving the enrichment or purity of biological samples with the probes.

### BACKGROUND OF THE INVENTION

Cells are composed of different types of biological molecules (biomolecules). The biomolecules in the cells interact with neighbor biomolecules in the subcellular environment to form complexes, organelles, or other assemblies and to carry out various cell functions. Characterizing the subcellular environment, within which biomolecules interact with one another, and how the biomolecules function together, is very challenging. Biomolecules are small, and they exist in a cell environment with tens of millions of other molecules. The interactions between neighboring biomolecules are frequently weak, and techniques used to study biomolecules disrupt their interactions. While techniques such as yeast two-hybridization assays and more recently proximity labeling have advanced our understanding of the cell environment, these techniques suffer from various limitations such as nonspecific binding, slow reaction times, and disruption of the natural cell environment, resulting in false positives and missed interactions. What is needed are better tools for determining naturally occurring biomolecule interactions.

Previous work has provided a photoreactive probe (see International Publication No. WO 2023/196986 A1). Compared to traditional technologies, this photoreactive probe can efficiently and accurately capture target biomolecules, enabling further analysis of interactions between biomolecules. Before conducting an analysis, such as mass spectrometry analysis, it can be helpful to purify the biological sample labelled with the photoreactive probe. However, the part of the probe that can be used for purification is the tag moiety, especially the commonly used biotin tag, which has low specificity. Meanwhile, biological samples already contain a large amount of biotin, which can easily lead to incomplete purification and have adverse effects on the analysis results. Provided herein are new photoreactive probes to address these and other problems, such as to improve the purification level of biological samples before analysis.

### SUMMARY OF THE INVENTION

The biotin tag is primarily used for enrichment of the targeted subpopulation for subsequent mass spectrometry (MS) analysis or other analysis. A limitation of these strategies is that mass spectrometry analysis does not easily discriminate unlabeled contaminants from the labeled protein subpopulation under study. To solve this or other problems, provided herein is a novel probe which has multiple functions, including proximity labelling achieved by a tag moiety, a photoreactive group for spatially interacting with the biomolecules of interest, and a phosphate group or a phosphonate group for efficient enrichment of labeling biomolecules for subsequent MS analysis or other analysis. This disclosure also provides a pull-down and purification method using the probes of the invention to enrich the labelling biomolecule achieved by two kinds of affinity matrix capturing the tag moiety and the phosphate group or the phosphonate group respectively. Also, the method may also enable isolation of low-abundant proteins and/or reduction of sample complexity for proteomic analysis.

One aspect of the disclosure provides a reactive probe including a chemical structure (I):

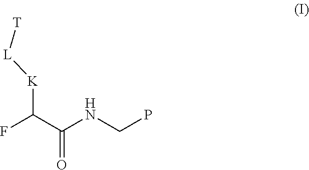

wherein P is either a phosphate group or a phosphonate group; K is a connecting group including —(CH₂)ₘ—Y—, wherein m is an integer ranging from 0 to 6, Y is selected from the group consisting of

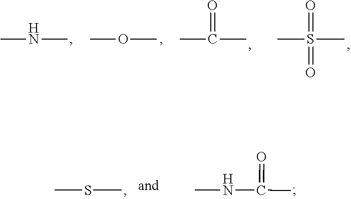

L is a linker between K and T; F is a reactive group; and T is a tag moiety.

In these and other embodiments of the reactive probe, the tag moiety is selected from the group consisting of a biotin derivative, a click chemistry tag, a CLIP-tag, a digoxigenin tag, a HaloTag, a peptide tag, an RNA molecule, a small molecule, a nucleic acid molecule, a chromogen, a fluorophore, a carboxyl group, a fluorescent in situ hybridization (FISH) probe, and a SNAP-tag.

In these and other embodiments of the reactive probe, the linker L has fewer than 24 atoms in a continuous chain.

In these and other embodiments of the reactive probe, the linker L is a covalent bond, such that the connecting group K and the tag moiety T directly bind to one another

In these and other embodiments of the reactive probe, the linker L is

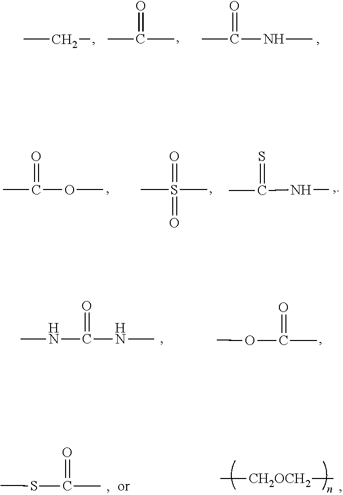

wherein n is an integer ranging from 1 to 6.

In these and other embodiments of the reactive probe, the connecting group K has no more than 15 atoms in a continuous chain.

In these and other embodiments of the reactive probe, the connecting group K can include —(CH₂)ₘ—Y—, wherein m is selected from 0, 1, 2, or 3, and Y is

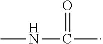

In these and other embodiments of the reactive probe, the tag moiety can be a click chemistry moiety selected from the group consisting of alkyne-based moiety and azide-based moiety.

In these and other embodiments of the reactive probe, the click chemistry moiety can include the moiety of

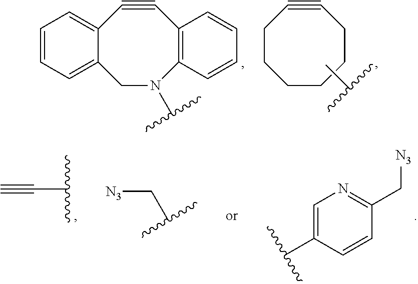

In these and other embodiments of the reactive probe, the tag moiety can be a biotin derivative selected from the group consisting of

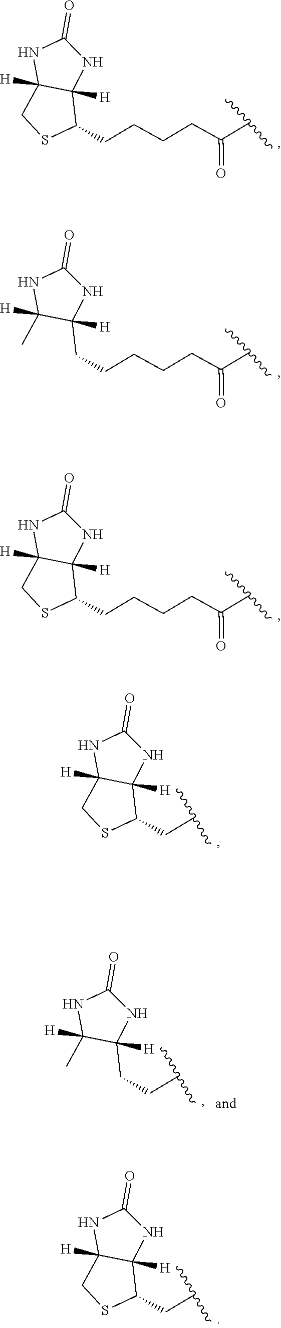

In these and other embodiments of the reactive probe, the reactive group can include a phenolic group, an aryl azide moiety, a benzophenone, a phenoxyl radical trapper, a light-activated warhead, a nucleobase-specific psoralen, or a diazirine.

In these and other embodiments of the reactive probe, the reactive group can include the phenolic group, and the phenolic group includes the moiety of

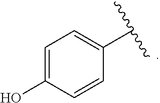 [SMILES: *c1ccc(O)cc1]

In these and other embodiments of the reactive probe, the reactive group can include the aryl azide moiety, and the aryl azide moiety can include one or more of

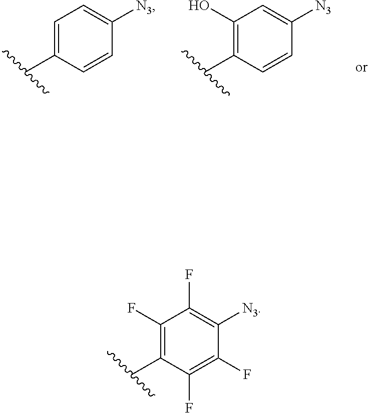 [SMILES: *c1ccc(N=[N+]=[N-])cc1 ; *c1ccc(N=[N+]=[N-])cc1O ; *c1c(F)c(F)c(N=[N+]=[N-])c(F)c1F]

In these and other embodiments of the reactive probe, the reactive group can include the diazirine and the diazirine can include the moiety of

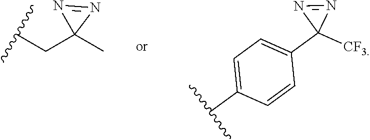

In these and other embodiments of the reactive probe, the reactive group can include the benzophenone, and the benzophenone can include the moiety of

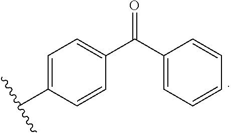 [SMILES: *c1ccc(C(=O)c2ccccc2)cc1]

In these and other embodiments of the reactive probe, the reactive group can include the light-activated warhead, and the light-activated warhead can include the moiety of nucleobase-specific 3-cyanovinylcarbazole nucleoside (CNVK), including the moiety of

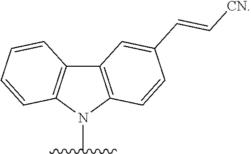 [SMILES: *n1c2ccccc2c2cc(/C=C/C#N)ccc21]

In these and other embodiments of the reactive probe, the reactive group can include the nucleobase-specific psoralen, and the nucleobase-specific psoralen can include the moiety of

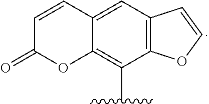 [SMILES: *c1c2occc2cc2ccc(=O)oc12]

In these and other embodiments of the reactive probe, the reactive group can include the phenoxyl radical trapper and the phenoxyl radical trapper can include the moiety of

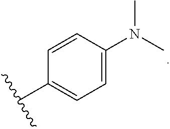 [SMILES: *c1ccc(N(C)C)cc1]

In these and other embodiments of the reactive probe, the reactive probe can be selected from the group consisting of

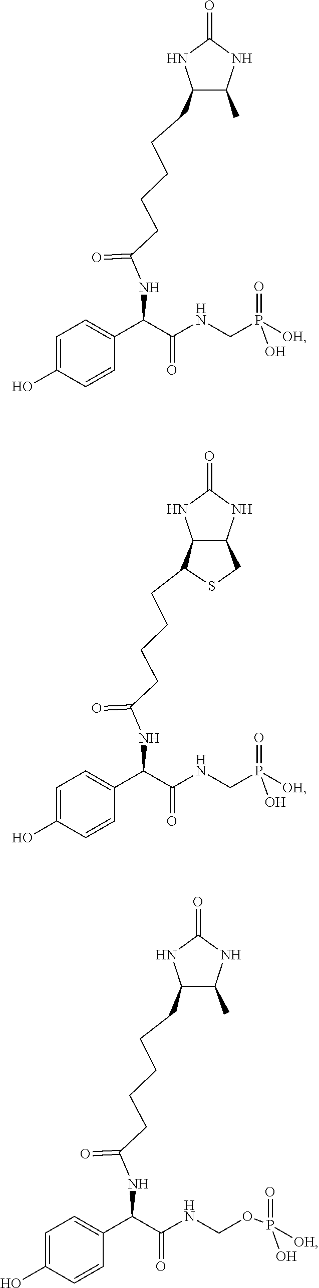

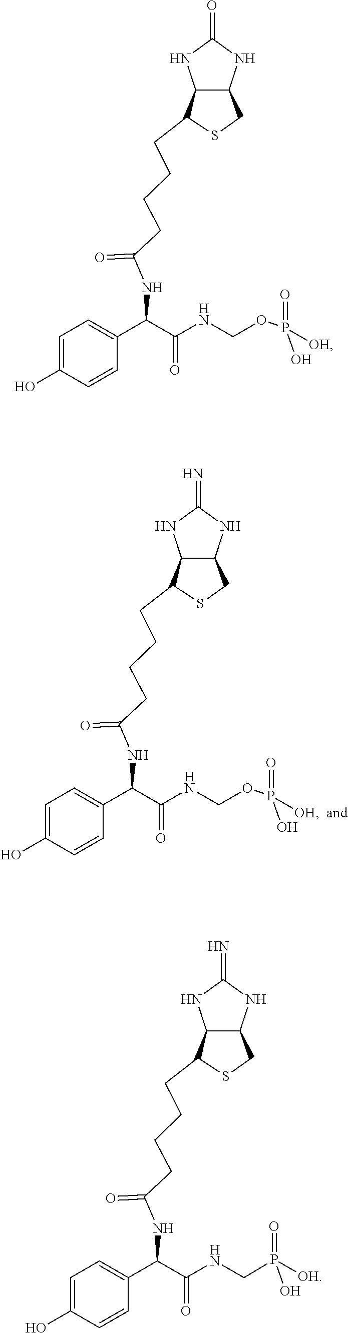

Another aspect of the disclosure provides a purification and pull-down method for chemical reaction labelling, the method can include the steps of: (a) delivering the reactive probe as claimed in any one of claims 1-18 to a biological sample; (b) activating, in a selected region of interest, the reactive probe to have a free radical to thereby generate an activated reactive probe; (c) forming, based on the free radical, a covalent bond between the activated reactive probe and a tyrosine of the biological sample; (d) contacting the biological sample with a first affinity matrix to thereby extract the biological sample labelled with the reactive probe; (e) eluting, from the first affinity matrix, the biological sample labelled with the reactive probe; (f) contacting the biological sample with a second affinity matrix to thereby extract from the biological sample labelled with the reactive probe; and (g) eluting, from the second affinity matrix, the biological sample labelled with the reactive probe.

In these and other methods, the method can further include a step of delivering an enzyme to the biological sample so as to activate the reactive probe to have the free radical. In these and other methods, step of delivering an enzyme to the biological sample can follow step (b) immediately or step (c) immediately and can precede other subsequent steps.

In these and other methods, the enzyme can include a peroxidase or a biotin ligase.

In these and other methods, the method can further include the steps of delivering a ruthenium-based compound to a location of the biological sample; and illuminating the region of interest of the biological sample to activate the ruthenium-based compound, wherein the activated ruthenium-based compound causes the reactive probe to have the free radical. The steps of delivering a ruthenium-based compound and illuminating the region of interest can be performed before performing step (a).

In some of these and other methods, the first affinity matrix can be a metal affinity matrix, and the second affinity matrix can include affinity beads which interact with the tag of the reactive probe labelling the biological sample. In some other of these and other methods, the first affinity matrix can be affinity beads which interact with the tag of the reactive probe labelling the biological sample, and the second affinity matrix can be a metal affinity matrix. The first affinity matrix can be used for purification first, and then the second affinity matrix can be used for purification. Alternatively, the second affinity matrix can be used for purification first, followed by the first affinity matrix for purification.

In these and other methods, the metal affinity matrix can be one or more of TiO₂ beads, a TiO₂ column, immobilized metal affinity chromatography (IMAC) beads, or an immobilized metal affinity column.

In these and other methods, the P of the reactive probe can form an ionic bond with the metal affinity matrix so as to pull-down the biological sample labelled with the reactive probe.

In these and other methods, the second affinity matrix can include affinity beads, and the affinity beads can interact with the tag of the reactive probe labelling the biological sample.

In these and other methods, the method can further include a step of using an alkaline phosphatase to dephosphorylate the biological sample. The step of using an alkaline phosphatase can be performed between any two method steps or can be performed before performing step (a).

In these and other methods, the method further can further include a step of digesting the biological sample labelled with the reactive probe bound on the first affinity matrix or the second affinity matrix so as to generate digested peptides labelled with the reactive probe bound on the first affinity matrix or the second affinity matrix. In these and other methods, the step of digesting the biological sample can be performed before step (d), between steps (d) and (f), or after step (f).

In these and other methods, the method can further include a step of digesting the biological sample with a protease so as to generate digested peptides labelled with the reactive probe. In these and other methods, the protease includes trypsin.

In these and other methods, the method can further include a step of analyzing the digested peptides labelled with the reactive probe through liquid chromatography mass spectrometry.

In general, in one embodiment, a purification and pull-down kit, including: the reactive probe as described above or elsewhere herein; and a ruthenium-based photocatalyst.

The disclosure herein provides a probe as shown in formula (I), which introduces the P moiety in its structure. The P moiety (which can be a phosphate group or a phosphonate group) can be connected to a metal affinity matrix for elution and purification. By introducing the P moiety, the biological sample can be purified twice before analysis (another purification is achieved through the interaction between the tag moiety and the affinity bead). The probes, methods and kits herein may advantageously improve purification quality, such as by reducing incomplete purification otherwise caused by the presence of background biotin in a biological sample. The probes, methods and kits herein may advantageously provide more accurate biomolecule analyses.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows a schematic depiction of a system useful for photoselective spatial tagging and proximity labeling of cells or biological sample on a substrate.

FIG. 2A shows a schematic illustration of a reactive probe.

FIG. 2B shows a schematic illustration of a photoreactive probe with a ruthenium-based compound.

FIG. 2C schematically illustrates a photoreactive probe and a reactive probe during use.

FIG. 2D schematically illustrates a proximity labeling system that can be used to label biomolecules in a small region of interest using the probes shown in FIGS. 2A and 2B.

FIG. 2E shows a schematic illustration comparing the results of direct photochemical labeling with photo-assisted enzymatic proximity labeling using the photoreactive probe and the reactive probe described herein to label biomolecules near the location where the photoreactive probe attached.

FIG. 2F schematically illustrates a method of enriching biomolecules labelled with a reactive probe in a pull-down system.

FIG. 2G schematically illustrates another method of enriching molecules labelled with a reactive probe in a pull-down system.

FIG. 2H schematically illustrates a method of enriching peptides labelled with a reactive probe with a pull-down system.

FIG. 2I schematically illustrates a method of enriching peptides labelled with a reactive probe with a pull-down system.

FIGS. 3A-1 to 3G show examples of photoreactive compounds that can be used in the photoreactive probes described herein.

FIGS. 3A-1 to 3A-14 show examples of ruthenium-based photocatalysts.

FIG. 3B shows examples of rose Bengal derivatives that can be included in the photoreactive probes described herein.

FIGS. 3C-1 to 3C-2 shows examples of fluorescein derivatives that can be included in the photoreactive probes described herein.

FIG. 3D shows an example of a methylene blue derivative that can be included in the photoreactive probes described herein.

FIG. 3E-1 to 3E-2 shows examples of lumiflavin derivatives that can be included in the photoreactive probes described herein.

FIG. 3F-1 to 3F-2 shows examples of riboflavin and flavin derivatives that can be included in the photoreactive probes described herein.

FIG. 3G shows examples of pterin derivatives that can be included in the photoreactive probes described herein.

FIGS. 4A-4L shows examples of linking groups that can be used as linkers in the reactive probes described herein.

FIGS. 5A-1 to 5E show examples of tag or T portions that can be used in the reactive probes described herein.

FIGS. 5A-1 to 5A-5 show examples of click chemistry moieties that can be used as tags in the reactive probes described herein.

FIGS. 5B-1 to 5B-6 show examples of biotin derivatives that can be used as tags in the reactive probes described herein.

FIG. 5C shows an example of a digoxigenin moiety that can be used as a tag in the reactive probes described herein.

FIG. 5D shows an example of a peptide tag that can be used in the reactive probes described herein.

FIG. 5E shows an example of a snap tag that can be used in the reactive probes described herein.

FIGS. 6A-1 to 6F shows examples of reactive groups that can be used in the reactive probes described herein.

FIGS. 7A-7L shows examples of amino acids that can be covalent bonded with the reactive probes described herein.

FIGS. 8A-8F shows examples of the structures of the reactive probes described herein.

FIG. 9 shows examples of the amide coupling reaction of the reactive probes with an antibody as described herein.

FIG. 10A shows an example of a proposed process for use in synthesizing the reactive probes disclosed herein.

FIG. 10B shows proposed methods for use in synthesizing the reactive probes disclosed herein.

### DETAILED DESCRIPTION OF THE INVENTION

The embodiments of the invention will be apparent from the following detailed description, which proceeds with reference to the accompanying drawings, wherein the same reference characters relate to the same elements.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

As used in the description herein and throughout the claims that follow, the meaning of “a”, “an”, and “the” includes plural reference unless the context indicates otherwise. Also, as used in the description herein, the meaning of “in” includes “in” and “on” unless the context indicates otherwise.

The ranges set forth herein may be interpreted as being inclusive of their endpoints, and open-ended ranges may be interpreted to include only commercially practical values. Similarly, lists of values may be considered as inclusive of intermediate values unless the context indicates the contrary. The recitation of ranges of values herein is merely intended to serve as a shorthand method of referring individually to each separate value falling within the range.

Methods described herein can be performed in any suitable order unless otherwise indicated herein or indicated otherwise by context. The use of examples, or exemplary language (e.g., “such as”), provided with respect to certain embodiments herein is intended to better illuminate the invention and does not pose a limitation on the scope of the invention otherwise claimed.

### ABBREVIATIONS AND DEFINITIONS

The term “antibody” refers to immunoglobulin and related molecules and includes monoclonal antibodies, polyclonal antibodies, monomers, dimers, multimers, multispecific antibodies (e.g., bispecific antibodies), heavy chain only antibodies, three chain antibodies, single chain Fv, and nanobodies. An antibody may be a polyclonal or monoclonal and/or may be a recombinant antibody. Antibodies may be murine, human, donkey, goat, humanized, chimeric, or derived from other species. As used herein, when an antibody or other entity “specifically recognizes” or “specifically binds” a target (e.g., an antigen or epitope), it can preferentially recognize the target (antigen or epitope) in a complex mixture of proteins and/or macromolecules and bind to the target (antigen or epitope) with affinity, which affinity is substantially higher than to other entities not displaying the antigen or epitope. A primary antibody binds to specific antigen. A secondary (tertiary, etc.) antibody binds specifically to another antibody and typically to a class or subclass of antibodies, usually through the Fc domain on the other antibody.

The term “bait molecule” refers to a molecule that specifically interacts with a (bio)molecule of interest, which (bio)molecule of interest may be referred to as a target (or prey). Examples of bait molecules include an antibody, CLIP-tag, a drug, a nucleic acid, a fluorescent in situ hybridization (FISH) probe, protein A, protein G, protein L, protein A/G, protein A/G/L, another small molecule, and a SNAP-tag.

The term “biotin derivative” refers to a biotin moiety, including biotin and variations of biotin, such as biotin with an open ring or substitutions. Typically, a biotin derivative is easily detectable with a biotin-binding entity or protein, such as avidin, neutravidin, or streptavidin. Neutravidin is a chemically modified avidin without glycosylation. A streptavidin/neutravidin-biotin bond is a very strong, non-covalent bond. The bond has been reported to have a dissociation constant of Kd=10⁻¹⁵ mol/L.

The term “click chemistry” refers to a chemical approach that easily joins molecular building blocks. Typically, click chemistry reactions are efficient, high-yielding, reliable, create few or no byproducts, and are compatible with an aqueous environment or without an added solvent. An example of click chemistry is cycloaddition, such as the copper(I)-catalyzed [3+2]-Huisgen 1,3-dipolar cycloaddition of an alkyne and azide leading to the formation of 1,2,3-triazole or Diels-Adler reaction. Click chemistry also includes copper free reactions, such as a variant using substituted cyclooctyne (see e.g., J. M. Baskin et al., Proc. Natl. Acad. Sci. U.S.A. 2007 Oct. 23, 104 (43), 16793-16797.) Other examples of click chemistry are nucleophilic substitutions; additions to C—C multiple bonds (e.g., Michael addition, epoxidation, dihydroxylation, aziridination); and nonaldol-like chemistry (e.g., N-hydroxysuccinimide active ester couplings). Click chemistry reactions can be bioorthogonal reactions, but do not need to be.

The term “instructional material” includes a publication, a recording, a diagram, a link, or any other medium of expression which can be used to communicate the usefulness of one or more compositions or methods of the disclosure for its designated use. The instructional material of a kit of the invention may, for example, be affixed to a container which contains the composition or components or be shipped together with a container which contains the composition or components. Alternatively, the instructional material may be shipped or otherwise delivered separately from a container with the intention that the instructional material and a composition or component be used cooperatively by the recipient.

The term “label” refers to a molecule which produces or can be induced to produce a detectable signal. In some embodiments, a label produces a signal for detecting a neighboring molecule. Examples of labels that can be used include avidin labels, neutravidin labels, streptavidin labels to detect a biotin tag.

The term “labelling” refers to a first moiety interacting with a second moiety and one of the moieties includes a label. Typically, the first and second moieties are in physical contact with one another.

The term “linker” refers to a structure which connects two or more substructures. A linker may have one (or more than one) uninterrupted chain of atoms extending between two substructures. The atoms of a linker are connected by chemical bonds, which in some embodiments are covalent bonds. In some variations, a linker may include two (or more than two) moieties joined specifically together, such as by hybridization therebetween. The moieties of a linker joined by hybridization are connected by chemical bonds, typically non-covalent bonds (e.g., hydrogen bonds).

The term “mass spectrometer” refers to an instrument for measuring the mass-to-charge ratio of one or more molecules in a sample. A mass spectrometer typically includes an ion source and a mass analyzer. Examples of mass spectrometers includes matrix assisted laser desorption ionization (MALDI), continuous or pulsed electrospray (ES) ionization, ionspray, magnetic sector, thermospray, time-of-flight, and massive cluster impact mass spectrometry.

The term “mass spectrometry” refers to the use of a mass spectrometer to detect gas phase ions.

The term “mass spectrometry analysis” includes linear time-of-flight (TOF), reflectron time-of-flight, single quadruple, multiple quadruple, single magnetic sector, multiple magnetic sector, Fourier transform, ion cyclotron resonance (ICR) or ion trap.

The term “phosphate group” refers to a derivative of phosphoric acid (e.g., H₃PO₄), such as a phosphate ester having a —COO— group that is part of a reactive probe. A phosphate group (or a chemical molecule or structure containing a phosphate group) can be in the form of one or more of an acid, a salt (e.g., lithium salt, sodium, potassium, etc.), and a phosphate ester (RₓH₃₋ₓPO4, wherein x=1 for monoester, x=2 for diester, and x=3 for triester).

The term “phosphonate group” refers to a chemical group containing a phosphorus (P) with the phosphorus bonded to a carbon (C), such as to a carbon atom of a reactive probe. In a phosphonate group, the phosphate is typically bonded to one or more (two or more, three) oxygen atoms. A phosphonate group may sometimes be represented herein by one or both of the chemical formula (i) —P(O)(OH)₂ and (ii) —PO(OR¹)(OR²) and include salts thereof. Examples of R¹ and R² independently include hydrogen (H), alkyl groups, and aryl groups and R¹ and R² can be the same or can be different. A phosphonate group (or a chemical molecule or structure containing a phosphonate group) can be in the form of a salt (e.g., lithium, sodium, potassium, etc.) or an ester of phosphorus acid.

The term “photoactivated” or “light activated” refers to excitation of atoms by means of radiant energy (e.g., by a specific wavelength or wavelength range of light, UV light, etc.). In some examples, a photoactivated catalyst promotes covalent bond formation between a tag-bearing phenol and an amino acid.

The term “photoreactive moiety” refers to a functional moiety, which, upon exposure to light (e.g., a specific wavelength or wavelength range of light, UV light, etc.) becomes activated. In some examples, a photoreactive moiety may promote covalent bond formation between a probe and an amino acid or another molecule. A photoreactive

The term “photoreactive probe” refers to a probe having a photoreactive moiety. A probe is a molecule configured to interact with (bind to) a target. Photoreactive probes typically have high affinity for their intended target and may also have selectivity over closely related target proteins. An example of a photoreactive probe is a secondary antibody with an attached photoreactive moiety. The secondary antibody is configured to bind to a primary antibody. Another example of a photoreactive probe is a primary antibody with an attached photoreactive moiety.

The term “protease” refers to an enzyme that catalyzes proteolysis, typically by hydrolysis of peptide bonds. Examples of proteases include aspartic proteases, cysteine proteases, glutamic proteases, serine proteases, threonine proteases, and metalloproteases, such as chymotrypsin, elastase, human rhinovirus 3C (HRV 3C), pepsin, thermolysin, tobacco etch virus (TEV) protease, trypsin, and trypsin-like protease. Some proteases may be useful for particular applications. Some proteases (such as trypsin) may be relatively available and inexpensive.

The term “proximity molecule” or neighboring molecule refers to a molecule that is near another molecule. A proximity molecule or neighbor molecule may bound to the molecule (e.g., covalently or non-covalently) or may be close by but not bound to the molecule.

The term “reactive group” refers to a group or moiety which can be activated in responsive to a specific stimulus. Typically, once activated, a reactive group can bind a molecule in a biological sample. An example of a reactive group is a light-activated warhead, and an example of a specific stimulus is light.

The term “small molecule” refers to low molecular weight molecules that include carbohydrates, drugs, enzyme inhibitors, lipids, metabolites, monosaccharides, natural products, nucleic acids, peptides, peptidomimetics, second messengers, small organic molecules, and xenobiotics. Typically, small molecules are less than (about) 1000 molecular weight. In some examples, small molecules are less than (about) 500 molecular weight.

The term “secondary antibody” refers to an antibody that specifically recognizes a region of another antibody. A secondary antibody generally recognizes the Fc region of a particular isotype of antibody. A secondary antibody may also recognize the Fc from one or more particular species.

The term “tag” refers to a functional group, compound, molecule, substituent, or the like, that can enable detection of a target molecule. A tag can provide or enable production of a detectable biological or physiochemical signal that allows detection by, e.g., absorbance, chemiluminescence, colorimetry, fluorescence, luminescence, magnetic resonance, phosphorescence, radioactivity. A detectable signal provided due to the tag can be directly detectable due to a biochemical or physiochemical property of a tag moiety (e.g., a fluorophore tag) or indirectly due to a tag interaction with another compound or agent. Typically, a tag is a small functional group or small organic compound. In some embodiments, a tag is a biotin derivative and can be a biotin conjugate. In some embodiments, the employed tag has a molecular weight of less than about 1,000 Da, less than about 750 Da, less than about 500 Da or even smaller.

The term “tagging” refers to the process of adding a tag to a functional group, compound, molecule, substituent, or the like. Typically, tagging enables detection of a target molecule.

The term “tyramide signal amplification” refers to a catalyzed reporter deposition (CARD). Tyramide signal amplification is an enzyme-mediated detection method that utilizes catalytic activity of an enzyme (e.g., horseradish peroxidase (HRP)) to catalyze inactive tyramide molecules to highly active tyramide molecules ready to be deposited. Each enzyme can catalyze many tyramide molecules (amplification) and amplification can take place in the presence of low concentrations of hydrogen peroxide (H₂O₂) to activate phenolic compounds (tyramide) for phenoxy radical reaction with nucleic acids or proteins (such as on tyrosine amino acids). In some embodiments herein, tyramide can be bound to (e.g., conjugated with) a biotin or biotin derivative.

The practice of the techniques described herein may employ, unless otherwise indicated, conventional techniques and descriptions of chemistry, biochemistry, cell biology, immunology, molecular biology (including cell culture, recombinant techniques, sequencing techniques) and organic chemistry technology which are explained in the literature in the field (e.g., Molecular Cloning: A Laboratory Manual, Fourth Edition, Green and Sambrook, eds., Cold Spring Harbor Laboratory Press, Cold Spring Harbor 2014, as well as corrections and updates thereto; John D. Roberts and Marjorie C. Caserio (1977) Basic Principles of Organic Chemistry, second edition. W. A. Benjamin, Inc., Menlo Park, CA.).

### Kits

Described herein are kits for practicing the methods described herein, e.g., for analyzing, tagging, and labeling biomolecules in order to perform a pull-down method or purification. A kit may include a probe, including any described herein. A kit will typically include instructional materials disclosing means for generating or modifying the one or more probes, such as attaching a bait molecule to photoreactive compound to prepare a photoreactive probe, applying the photoreactive probe to a sample, conjugating the bait molecule of the photoreactive probe to a prey molecule (in the sample), removing (washing away) unconjugated photoreactive probe, applying a reactive probe to the sample, photoactivating the photoreactive probe for bonding the reactive probe to a molecule of interest, removing (washing away) unbound reactive probe.

A kit may also include additional components to facilitate the particular application for which the probe is designed. Thus, for example, a kit may additionally contain components to facilitate detecting a sample and/or detecting a label (e.g., enzyme substrates for enzymatic labels, filter sets for detecting fluorescent labels, enzymes or associated detection reagents, including reagents for performing catalyzed reporter deposition (CARD) or signal amplification (e.g., avidin, neutravidin, streptavidin, HRP, tyramide, hydrogen peroxide, etc.). A kit may additionally include wash solutions, such as blocking agents, detergents, and/or salts (e.g., sodium chloride, potassium chloride, phosphate buffer saline (PBS)) for performing one or more steps (e.g., after sample fixation). A kit may include variations of wash solutions, such as concentrated wash buffers configured to be diluted before use or components to use for making one or more wash solutions and other reagents routinely used for the practice of a particular method. A kit may include fixatives and other sample preparation materials (e.g., ethanol, methanol, formalin, paraffin, etc.)

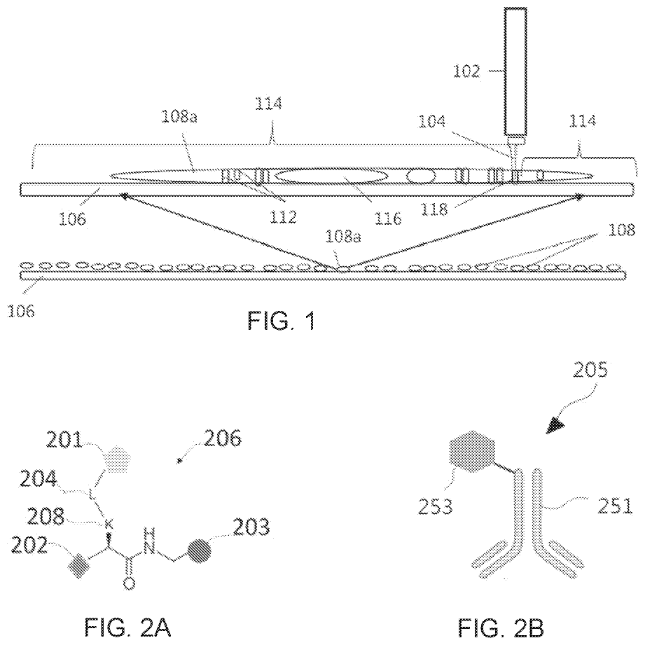

The reactive probes can advantageously be used with a microscope system, such as the systems described herein and in U.S. Pat. No. 11,265,449, to enable automatic labeling of cellular biomolecules proximal to a biomolecule of interest. The labelled molecules may be adjacent the biomolecule of interest or may be close-by but not adjacent, such as when intervening molecules are between the molecule of interest and cellular biomolecules for capture or analysis. Molecules that are close-by but not adjacent to a molecule of interest may be part of cell structure or otherwise contribute to a cell microenvironment of interest. FIG. 1 shows a schematic depiction of a system useful for photoselective spatial tagging and labeling. The bottom part of FIG. 1 shows substrate 106, such as a microscope stage or a carrier for cells 108, and a monolayer of a plurality of cells 108 disposed on the substrate. In some embodiments, the surface of an entire substrate, or a portion of the substrate, can be analyzed using an automated microscope system to identify a region of interest. For example, a sample can be stained or labelled to identify a region of interest. The top part of FIG. 1 shows an expanded view of cell 108a, one of the plurality of cells 108. The cell 108a has a nucleus 116 and a plurality of different types of organelles 112, such as cell membranes, mitochondria, ribosomes, and vacuoles. Microscope system 102 selectively shines narrow band of light 104 onto region of interest (ROI) 118 for analysis of the region of interest 118. The illumination can be selective, and large regions 114 of the cell and substrate may be not illuminated. As explained in more detail below, narrow band of light 104 activates a photoreactive probe to facilitate bonding or labeling between a photoreactive probe and a biological sample in only the region of interest 118. Multiple (e.g., at least 2, at least 5, at least 10, at least 100, at least 1000, at least 10,000, or more or fewer than these numbers of regions (e.g., less than 10,000, etc.) of interest can be illuminated.

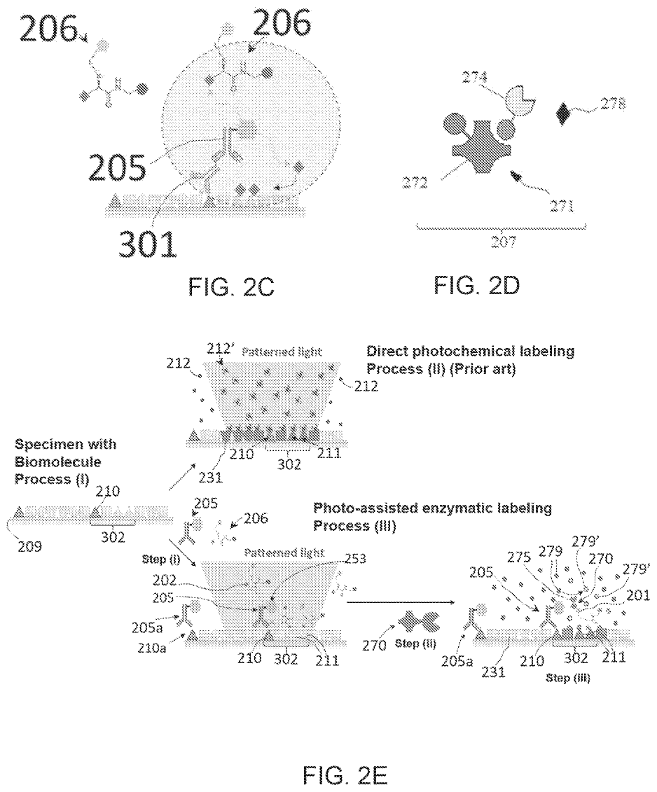

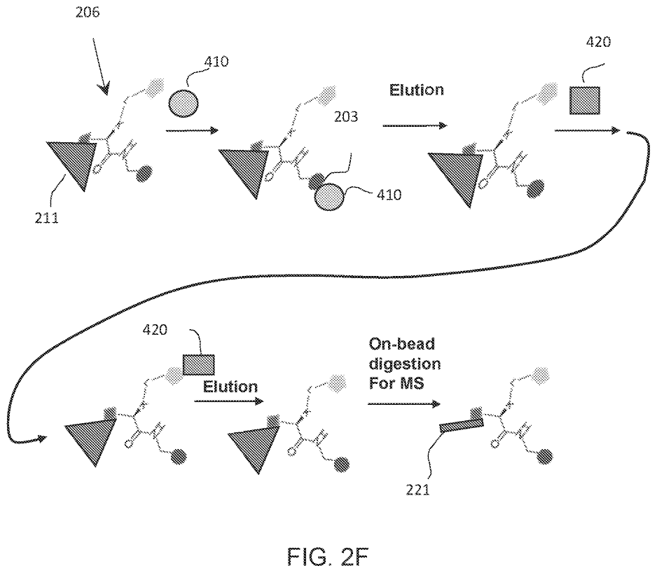

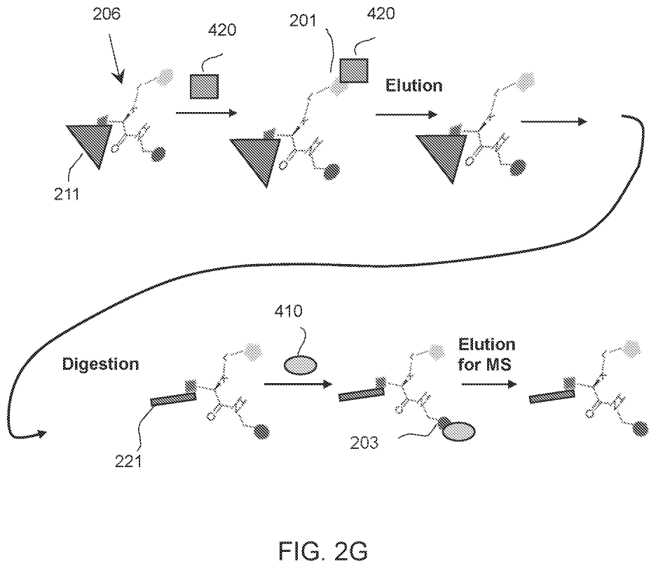

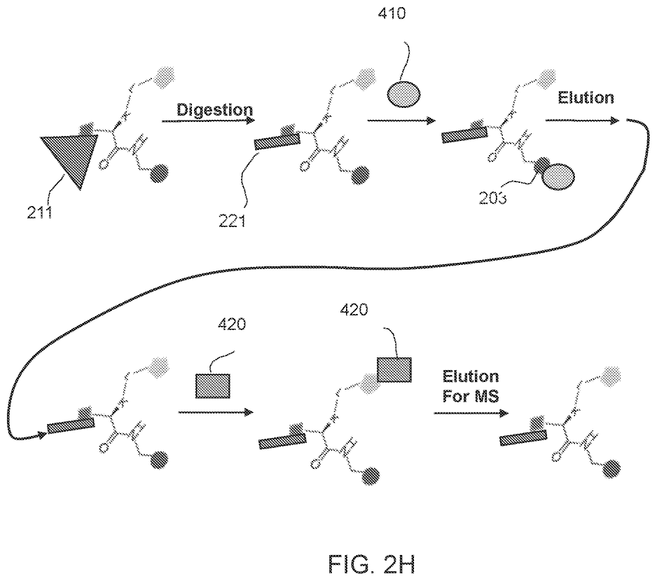

FIG. 2A schematically illustrates reactive probe 206 with tag 201, the reactive group 202, the P portion 203, the L portion 204, and the K portion 208. P portion 203 is connected to the rest of the reactive probe via a carbonyl group and an amide group. In some embodiments, reactive probe 206 shown in FIG. 2A can be represented by chemical structure (I):

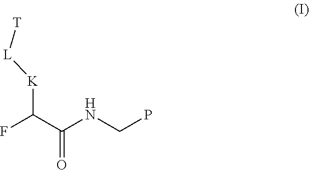

wherein, P portion 203 is either a phosphate group or a phosphonate group; K portion 208 is a connecting group including —(CH₂)ₘ—Y—, wherein m is an integer ranging from 0 to 6 (such as 0, 1, 2, 3, 4, 5 or 6), Y is selected from the group consisting of

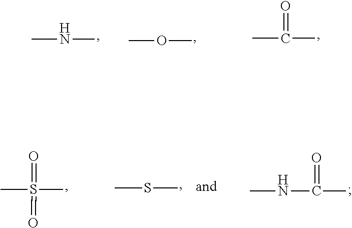

L portion 204 is a linker between K portion 208 and T portion (tag 201); F portion 202 is a reactive group; and T portion (tag 201) is a tag moiety. In some embodiments, the K portion is a connecting group including —(CH₂)ₘ—Y—, wherein m is selected from 0, 1, 2, or 3, and Y is

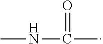

FIG. 2B schematically illustrates a photoreactive probe 205 with a bait molecule 251 conjugated with a photoreactive compound 253. The photoreactive compound 253 preconjugated with the bait molecule 251 to form the photoreactive probe 205. Although this example shows the bait molecule 251 as an antibody (such as a secondary antibody), any bait molecule as described herein can be used. FIG. 2C illustrates a substrate with sample thereon containing a plurality of molecules. The sample can be, for example, a cell sample, an organelle sample, a monolayer of cells, etc. A primary antibody is delivered to the sample and binds to a molecule of interest. FIG. 2C shows the primary antibody is now the target of interest, target biomolecule 301. Photoreactive probe 205 (photoreactive probe 205 is shown in detail in FIG. 2B and details of photoreactive probe 205 may be omitted in FIG. 2C or other figures for clarity) is delivered to the sample and allowed to detect and bind to target molecule 301 (shown in FIG. 2C). (In other examples, a target molecule of interest can be another structure, such as a carbohydrate, a lipid, a nucleic acid, or a protein, in the sample). FIG. 2C illustrates reactive probe 206 is added to the sample (reactive probe 206 is shown in detail in FIG. 2A and details of reactive probe 206 may be omitted in FIG. 2C and other figures for clarity). FIG. 2C illustrates that, upon selectively photoexcitation of the photoreactive probe 205, the photoreactive probe 205 activates the reactive group 202 of the reactive probe 206 so as the reactive probe 206 label the molecules near the target biomolecule 301. Although this example shows primary antibody as the target biomolecule 301, photoreactive probe 205 can detect and bind to any other target molecule (biomolecule) as described herein. The tag 201, the reactive group 202 and the P portion 203 of the reactive probe 206 can be detected using the methods described herein. For example, if the tag 201 of reactive probe 206 is a biotin derivative, it can be detected by using an avidin/streptavidin/neutravidin detection method. If the P portion 203 of reactive probe 206 is a phosphate group or a phosphonate group, it can be detected by using a metal affinity matrix method. If the reactive group 202 of reactive probe 206 is a phenol group, it can be detected using a Tyramide Signal Amplification (TSA) system, forming covalent bonds with tyrosine residues. Use of such methods may significantly reduce the background noise by means of the selective catalytic or photocatalytic probe, label cytosolic organelles with higher resolution and finer detail, and achieve subcellular proteomics.

FIG. 2D shows an example of a labeling system 207 that can be used with the reactive probe 206 shown in FIG. 2A to label biomolecules neighboring a target biomolecule of interest. The labeling system 207 includes a labeling complex 271 with a connector 272 and an enzyme or catalyst 274, and an additional subject probe 278. Although this example shows a fluorescent connector as the connector 272, a connector without fluorophore can be used. In some embodiments, the connector 272 can be avidin-dye conjugate, streptavidin-dye conjugate, neutravidin-dye conjugate or the like, and the enzyme or catalyst 274 can be tag-peroxidase configured to utilize peroxide (not shown) for activity. In this example, the tag 201 and the connector 272 recognize one another and conjugate. The enzyme or catalyst 274 activates the additional subject probe 278 and, once activated, the activated subject probe 278 (e.g., tyramide probe) can bind to and detectably label biomolecules in its vicinity. The additional subject probe 278 can include a tag and a reactive group and may be the same as or different from the reactive probe 206. Any tag and reactive group described herein for the reactive probe 206 also can be used as an additional subject probe 278.

FIG. 2E shows a comparison of a direct photochemical labeling (top, labelled Process II) and photo-assisted enzymatic labeling (bottom, labelled Process III) on a specimen with molecules (Process I) to label biomolecules in a small region of interest (ROI) 302. Process III illustrates use of the photoreactive probes 205, the reactive probes 206, and systems described herein. Prior to performing either Process II or Process III, a sample (e.g., a cell or tissue sample) containing a biomolecule of interest 210 (protein will be used herein by way of example, but other biomolecules could instead be analyzed) is analyzed and a region of interest 302 identified. The sample can be pretreated, such as fixed and stained. For example, a sample can be fixed and stained with a cell stain (e.g., hematoxylin and eosin (H &E); Masson's trichrome stain). A protein of interest can be identified by an immunofluorescent labelled antibody that recognizes the protein of interest delivered to the sample that binds to the protein of interest (or by other methods). Once the region of interest 302 is identified, a plurality of neighboring molecules within the region of interest 302 is analyzed. As illustrated in Process IL, the sample is treated with a direct photoreactive probe 212. Direct photoreactive probe 212 is widely available and patterned light is directed to the sample and activates direct photoreactive probe 212 to form activated direct photoreactive probe 212′ only in the region which has received patterned light. The activated direct photoreactive probe 212′ (black dots within a ring) is able to form complexes with other molecules in close vicinity (show by the shaded area in the top portion of FIG. 2E (Process II)). The activated direct photoreactive probe 212′ can diffuse and label neighbor molecules 211 near the molecule of interest 210. However, the labeling diameter (300-600 nm) of the region of photoactivation of the direct photoreactive probes 212 can be relatively large as the region of photoactivation is spatially determined by the diffraction limit of the light sources used. Additionally, since the direct photoreactive probe 212 (as shown in the top portion of FIG. 2E (Process II)) is free to diffuse, any proteins in the relatively large pathway of the patterned light can be labelled. Process II also shows activated direct photoreactive probe 212 can label molecules 231 far away from biomolecule of interest 210. The region labelled by the activated direct photoreactive probe 212′, or labelled or labelling precision, covers a region of about 300-600 nm. This region can include biomolecules that are not in sufficiently close proximity to biomolecule of interest 210 for some applications, and in some cases might lead to confusing, misleading, or unhelpful results.

In contrast, in Process III, shown on the bottom of FIG. 2E, the photoreactive probe 205 (which recognizes the biomolecule of interest 210) is delivered to the sample. As illustrated in FIG. 2E Step (i), similar as to Process IL, patterned light is also directed to the sample. However, here, the patterned light activates the photoreactive compound 253 of the photoreactive probe 205 bound to the molecule of interest 210. The activated photoreactive probe 205 can activate the reactive group 202 of the reactive probe 206 and the activated reactive probe 206 forms a covalent bond with molecules 211 or other moieties close by. In addition to patterned light directing a limited region of activation of the photoreactive probe 205, the readily accessible reactive probe 206 is only activated within a restricted catalyst radius shown in FIG. 2C, instead of undergoing long-range diffusion after activation. Thus, the reactive probe 206 becomes covalently bound to the neighbor molecules 211 within the region of interest 302. Step (i) (at the bottom of FIG. 2E) also shows how background or unwanted labeling can be reduced using the probes and methods described herein. In Step (i), a photoreactive probe 205a (the “a” indicates the probe is outside the region of patterned light) is attached to a molecule 210a (the “a” indicates the molecule is outside the region of patterned light); however, since the photoreactive probe 205a is outside the light delivery region, the photoreactive probe 205a is not activated in this region, resulting in no activation of the reactive probe 206 and no bonding between the reactive probe 206 and molecules outside the light delivery region.

Unbound reactive probes 206 can be washed away with wash solution. FIG. 2E Steps (ii) and (iii) illustrate further and additional labeling of molecules near the molecule of interest 210 using a neutravidin-peroxidase structure 270. A biotin derivative is used here as an example of a tag, peroxidase is used as an example of an enzyme 275, and peroxidase substrate (e.g., peroxide) is used here as an example of a subject probe 279. Neutravidin-peroxidase structure 270 and subject probe 279 (e.g., peroxidase substrate; peroxide) are delivered to the sample (which can be simultaneously or in succession). When the tag 201 of the reactive probe 206 is a biotin derivative, the neutravidin moiety of the neutravidin-peroxidase structure 270 conjugates with the tag 201 (biotin) of the reactive probe 205. Enzyme or catalyst 275 (e.g., peroxidase) activates the subject probe 279 (e.g., peroxidase substrate; peroxide) to form activated subject probe 279′ (e.g., a peroxide radical). As illustrated in FIG. 2E step iii, since the photoreactive probe 205 is attached to molecule of interest 210, the neighbor molecules 211 are labelled by reaction with the activated subject probe 279′, while the molecule 231 far away from the molecule of interest 210 is not labelled with any activated subject probe 279′. In some embodiments, other labeling systems can be used. For example, the labeling system 207 shown in FIG. 2D can be conjugated with the tag 201 of the reactive probe 206 and the enzyme or catalyst 274 shown in FIG. 2D activates the additional subject probe 278 to form the activated additional subject probe. In this case, the neighbor molecules 211 are labelled with activated subject probe 279′, while the more distant molecule 231 is not.

By photoselectively localizing the enzyme or catalyst 275, such as peroxidase, near the biomolecule of interest 210 and labelling the neighbor molecules 211 in the region of interest 302 using the tagging or labeling just described, the coupling reaction can be localized to a region as small as <100 nm. In some variations, a larger region (e.g., up to (about) 200 nm, up to (about) 300 nm, up to (about) 400 nm, up to (about) 500 nm, up to (about) 1 μm, up to (about) 2 μm, up to (about) 5 μm) could be labelled. Furthermore, some biomolecules of interest 210 in a sample have more than one region of localization and hence interact with different molecular complexes in different locations simultaneously. The photo-labeling can be performed successively in more than one location. For example, after applying light as shown in FIG. 2E, Process III and tagging/labelling the neighbor molecules as indicated, the light can be selectively applied to a second (third, fourth, etc.) location in the sample and this process can be repeated as many times as desired. In addition to labeling (depositing labels) to a relatively small number of neighbor molecules in a very small area of a sample, by using a microscope to direct the light and the probes described herein, and as explained herein, the process can also be performed with sufficiently mild or gentle treatments so that the cell architecture remains intact.

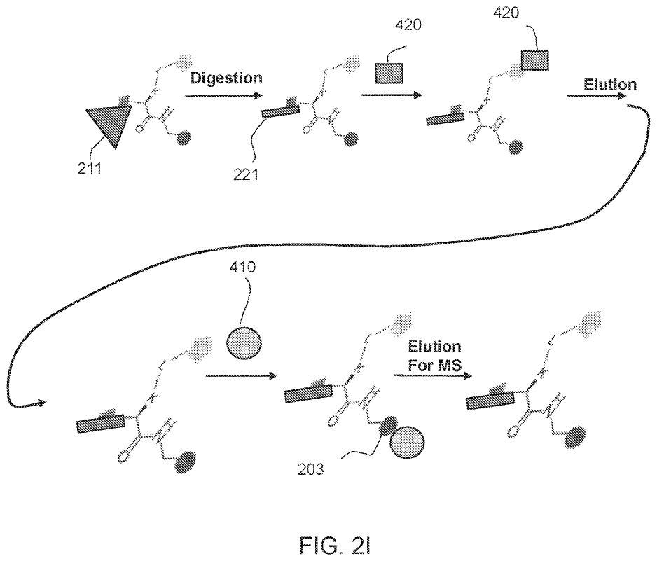

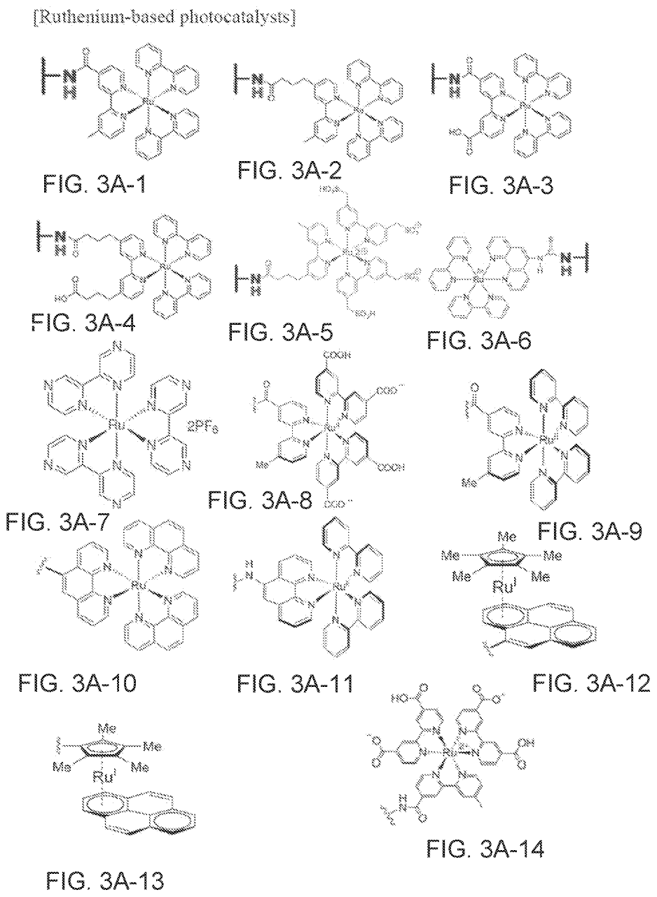

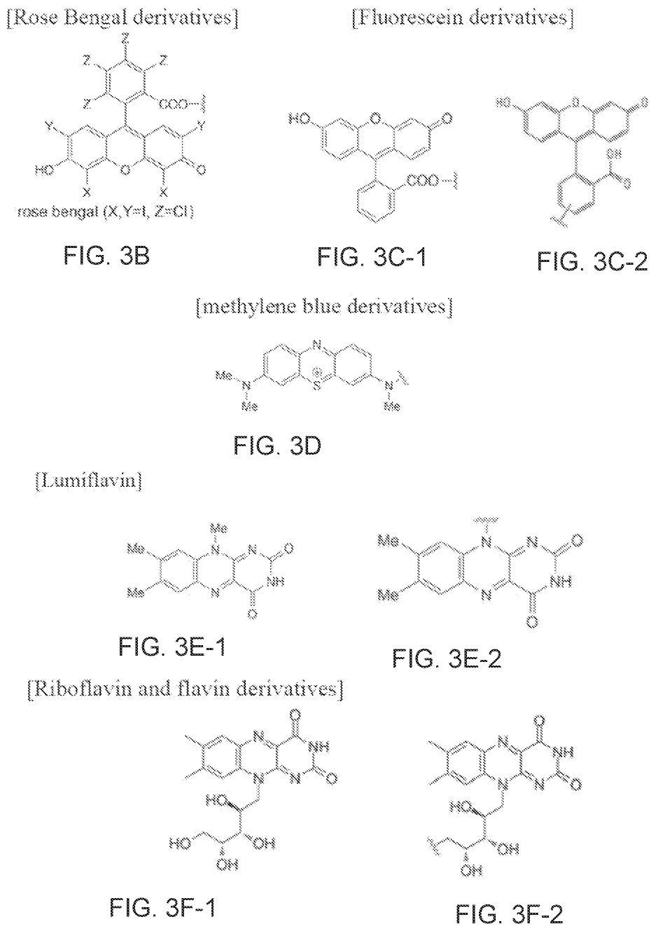

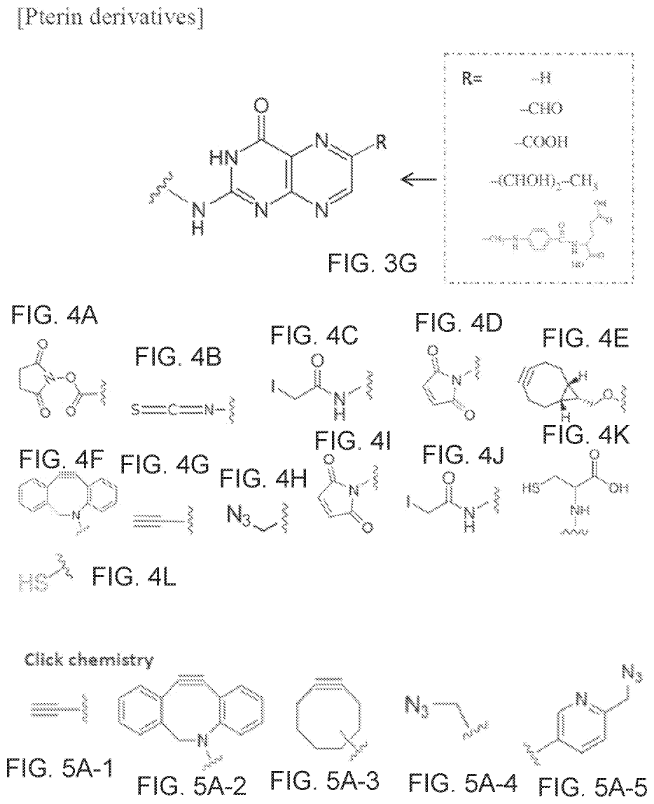

FIGS. 3A-1 to 3G show certain examples of photoreactive compounds that can be used in the photoreactive probes described herein. FIGS. 3A-1 to 3A-14 shows ruthenium-based photocatalysts. FIG. 3B shows Rose Bengal derivatives. FIGS. 3C-1 to 3C-2 show fluorescein derivatives. FIG. 3D shows methylene blue derivatives. FIG. 3E-1 to 3E-2 shows examples of lumiflavin derivatives. FIG. 3F-1 to 3F-2 shows examples of riboflavin and flavin derivatives. FIG. 3G shows examples of pterin derivatives. The selection of a particular photoreactive compound can depend on the desired wavelength and the types of the bait molecule. For example, the constituents of the photoreactive probe and constituents for the pre-probe analysis can be chosen so as to not interfere (or minimally interfere) with each other.

The reactive probe 206 represented by chemical structure (I) includes linker 204 shown in FIG. 2A. The linker 204 can be a linking group. Linker 204 be one or more of (or can be selected from the group consisting of)

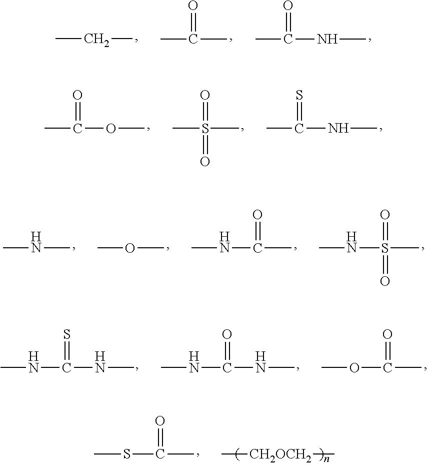

and a combination of at least two groups thereof, wherein n is an integer ranging from 1 to 6. In some embodiments, two or more than two of the linking groups can be chemically joined together to form a linker. In some embodiments, linker 204 of the reactive probe 206 may be a covalent bond (and not have a linking group) such that the connecting group K and the tag moiety bind directly to each other (the K portion 208 and T portion (tag 201) directly link together).

FIGS. 4A-4L shows examples of linking groups that can be used as linkers 204 in the reactive probes 206 described herein. For the L portion 204 of the reactive probe 206, some embodiments can include, for example, NHS-BCN, NHS-(PEG)n-BCN, NHS-DBCO, NHS-(PEG)n-DBCO, NHS-alkyne, NHS-(PEG)n-alkyne, NHS—N3, NHS-(PEG)n-N3, NHS-maleimide, NHS-(PEG)n-maleimide, NHS-iodoacetyl group, NHS-(PEG)n-iodoacetyl group, NHS-cysteine/thiol group, NHS-(PEG)n-cysteine/thiol group, maleimide-peptide/amino acid, maleimide-(PEG)n-peptide/Amino acid, maleimide-oligonucleotide, iodoacetyl-peptide/amino acid, iodoacetyl-(PEG)n-peptide/amino acid, iodoacetyl-oligonucleotide or derivatives, n each independently being an integer of 1-20. In some embodiments, a linking group of linker 204 includes the moiety of (PEG)n, peptide, amino acid, or oligonucleotide, and wherein n each independently is an integer of 1-20. Other examples of a polymeric linking group that may be useful for the reactive probes herein include polypropylene glycol, polyethylene, polypropylene, polyamides, and polyesters. The linking group of linker 204 can be a linear molecule with a chain of at least one or two atoms and can include more.

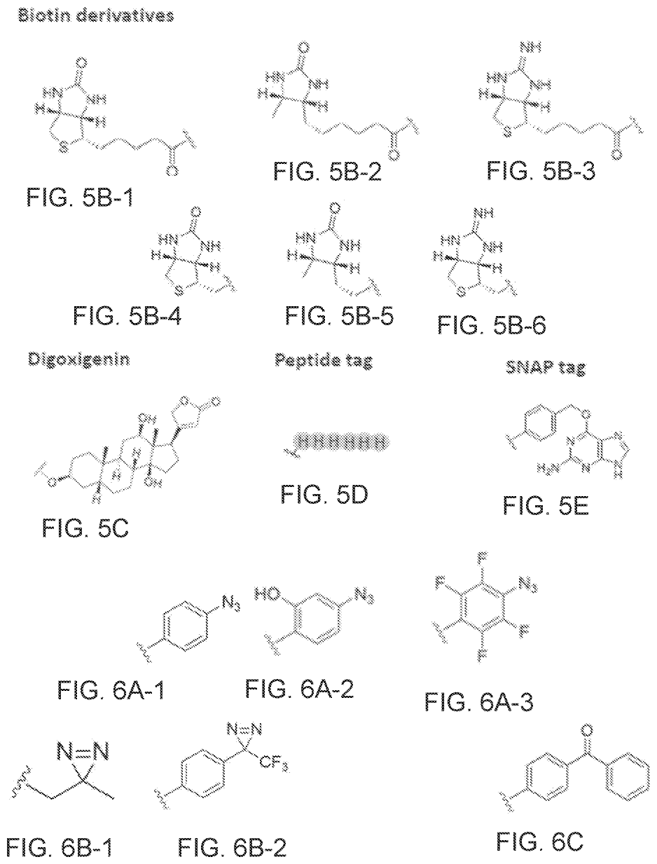

FIGS. 5A-1 to 5E show examples of tag 201 or T portion that can be used as tag 201 in the reactive probes 206 described in FIG. 2A. Tag 201 or T portion is configured to interact with a pull-down system for precipitation of the reactive probe 206 and complex including the reactive probe 206. FIGS. 5A-1 to 5A-5 shows examples of click chemistry tags that can be used as tag 201 in reactive probe 206. A click chemistry tag may be, for example, an azide-based moiety, or an alkyne-based moiety. FIGS. 5B-1 to 5B-6 shows examples of biotin derivatives that can be used as tag 201 in reactive probe 206. FIG. 5C shows a digoxigenin moiety tag. FIG. 5D shows a peptide tag. In particular, FIG. 5D shows a poly His tag with 6 histidines. However, a histidine tag could instead have fewer histidines or more histidines, such as 5 histidines or 7-10 histidines or more. FIG. 5E shows a SNAP-tag. In some instances, a CLIP-tag or HaloTag could be used. In some embodiments, the tag 201 or T portion is a nucleic acid molecule and may be useful to enrich an RNA binding protein or RNA binding protein complex so as to incorporate the tag into another molecule.

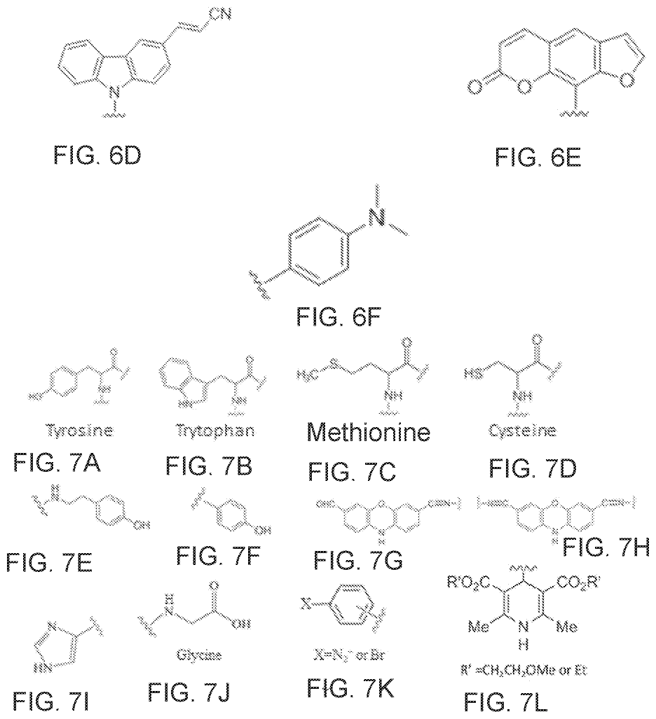

FIGS. 6A-1 to 6F show examples of the reactive group 202 that can be used in the reactive probes 206 described in FIG. 2A. The reactive group 202 is configured to detectably label molecules neighboring a target molecule of interest. The reactive group 202 that can be used with the reactive probes 206 includes a phenol, an aryl azide, a benzophenone, a phenoxyl radical trapper, a carboxyl group, a nucleobase-specific psoralen, a light-activated warhead, or a diazirine. FIGS. 6A-1 to 6A-3 shows examples of the moiety of the aryl azide. FIGS. 6B-1 to 6B-2 shows examples of the moiety of the diazirine. FIG. 6C shows examples of the moiety of the benzophenone. FIG. 6D shows examples of the light-activated warhead including the moiety of a nucleobase-specific 3-cyanovinylcarbazole nucleoside (CNVK). FIG. 6E shows examples of a moiety of the nucleobase-specific psoralen. FIG. 6F shows examples of the moiety of the phenoxyl radical trapper. In some embodiments, the carboxyl group of the reactive group 202 can be used for BioID system so as to conjugate with the substrate of biotin ligase. After activation, a reactive intermediate (such as a free radical) can be generated from the reactive group 202 and poised for covalent bond formation with amino acid in proximity as shown in FIGS. 7A-7L. The amino acid may be alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or the like.

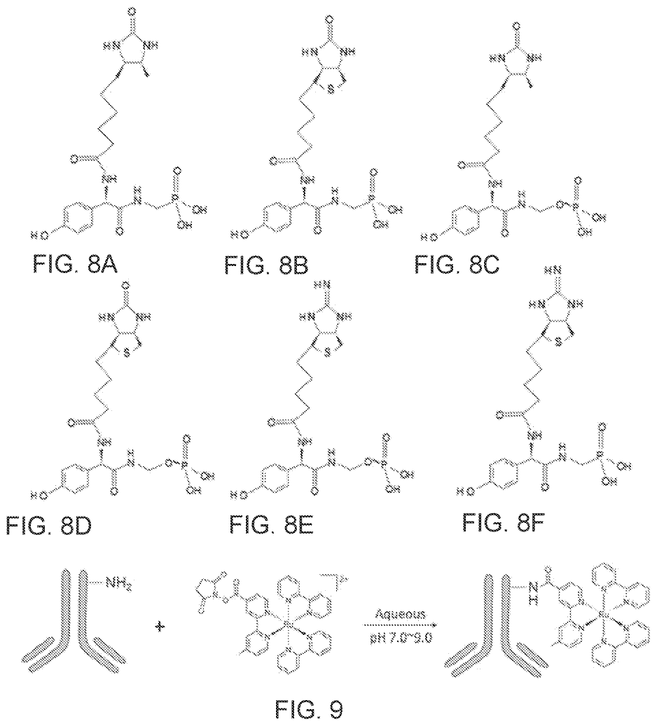

In some embodiments, the reactive probe 206 is selected from the group of probes shown in FIGS. 8A-8F. In these examples, the reactive group 202 is a phenol, which can be activated so as to label a biomolecule close by a molecule of interest, while the tag 201 is a biotin derivative and the P portion 203 is either a phosphate group or a phosphonate group. The tag 201 and the P portion 203 are used to pull-down or concentrate the protein labelled with the reactive probe 206.

In an implementation of photocatalytic ruthenium complex-antibody conjugate as a photoreactive probe, bis(2,2′-bipyridine)-4′-methyl-4-carboxybipyridine-ruthenium can be specifically conjugated to a secondary antibody through NHS-amide linkage that selectively hybridize to the primary antibody of the region of interest (ROI). [Ru(bpy)₃]²⁺ is a photocatalyst that can be excited by either single- or two-photon illumination around 425 nm and 780 nm respectively. The oxidized [Ru(bpy)₃]³⁺ can seize an electron from the phenolic hydroxyl group of biotin-phenol as a primary subject probe and generate a phenoxyl radical and proton to the tyrosine residues in proximity. To achieve a higher density of labeling for proteomic profiling, HRP amplification takes place to further covalently bind tyramide radicals to nearby tyrosine residues of the protein neighbors prior to streptavidin-enrichment and on-bead digestion. Finally, a subcellular/localized proteomic profile can be obtained by quantitative proteomics. The implementation is characterized by antibody-based ruthenium complex ([Ru(bpy)₃]²⁺) antibody conjugate as a selective photocatalytic probe for spatial and localized proteomics, identification of novel proteins of cellular organelles that are unable to fractionate or isolate by conventional methods, and size and morphological distinguishable labeling via single- or two-photon illumination.

Photoselective tagging and labeling as described herein can be performed in various types of samples, such as samples obtained from tissues, cells, or particles, such as from an entity (e.g., a human subject, a mouse subject, a rat subject, an insect subject, a plant, a fungi, a microorganism, a virus) or tissues samples or cell samples that are not from an organism, such as cell culture samples or artificial tissue scaffold samples (e.g., cultured laboratory cells, in vitro developed heart tissue, 3-d printed tissue, etc.). Samples for analysis using the probes, materials, and methods described herein can be living (live cells) or can be not living (e.g., fixed). A sample for tagging and labeling can include a monolayer sample, a multi-layer sample, a sample fixed to a substrate (e.g., a microscope slide), a sample not fixed to a substrate, a suspension of cells, or an extract, such as an in vitro cell extract, a reconstituted cell extract, or a synthetic extract. In some embodiments, a sample is not fixed (unfixed). Examples of probes useful for tagging live cells include those utilizing a small molecule or those sometimes referred to as self-labeling molecules (e.g., Clip-tag, Halo-tag, SNAP-tag). In some embodiments, a large number of cells can be automatically analyzed using the methods and materials described herein (e.g., at least about 1,000 cells, at least 10,000 cells, at least 100,000 cells, at least 1 million cells). In some embodiments, a smaller number of cells can be analyzed, such as no more than 1,000 cells, no more than 100 cells, or only a few cells or a single cell. In some embodiments a sample is fixed. For example, a cell or tissue sample may be fixed with e.g., acetic acid, acetone, formaldehyde (4%), formalin (10%), methanol, glutaraldehyde, or picric acid. A fixative may be a relatively strong fixative and may crosslink molecules or may be weaker and not crosslink molecules. A cell or tissue sample for analysis may be frozen, such as using dry ice or flash frozen, prior to analysis. A cell or tissue sample may be embedded in a solid material or semi-solid material such as paraffin or resin prior to analysis. In some embodiments, a cell or tissue sample for analysis may be subjected to fixation followed by embedding, such as formalin fixation and paraffin embedding (FFPE).

A concentration of the photoreactive probe can range from 0.1 ug/mL to 100 ug/mL, while a concentration of the reactive probe can range from 1 uM to 20 mM. The wavelength of light for activation of the photoreactive probe or photoselective tagging and labeling ranges in some embodiments from about 200 nm to about 1600 nm. In some embodiments, the wavelength of light for performing photoselective tagging and labeling ranges from about 700 nm to about 1600 nm (e.g., 780 nm) from a two-photon light source; or ranges from about 300 nm to about 650 nm (e.g., 455 nm) from a single-photon light source. The wavelengths used for photoactivation of the probe are typically different from the wavelengths used for imaging. In some embodiments, the activation of the photoreactive probe utilizes optical radiation (light) from around 300-450 nm, 550 nm for single photon activation or >720 nm for multiphoton activation. The particular wavelength depends on the particular photoreactive compound of the photoreactive probe.

### Methods

Also disclosed herein are pull-down and purification methods for chemical reaction labelling biomolecules for further analysis. The methods may be used to tag and/or label carbohydrates, lipids, nucleic acids, proteins, either alone or in combination. The methods may include the step of treating a biological sample with a reactive probe having the tag 201, the reactive group 202, the P portion 203, the L portion 204 and the K portion 208 and being configured to label the biological sample around a photoreactive probe. In some embodiments, the biological sample includes a plurality of cells and the photoreactive probes were delivered to a target biomolecule. In some embodiments, the biological sample includes a plurality of living cells. In some embodiments, the biological sample includes at least one, at least 100, at least 1000 or at least 10,000 live cells. In some embodiments, the biological sample includes cell extracts. In some embodiments, the photoreactive probe including the photoreactive compound which is coupled to the bait molecule through a chemical bond or a linker. In some embodiments, the photoreactive probe is configured to be illuminated so as to active the reactive probe. In some embodiments, the bait molecule of the photoreactive can include an antibody, a CLIP-tag, HaloTag, a SNAP-tag, protein A, protein G, protein L, protein A/G, protein A/G/L, immunoglobulin binding peptides, avidin, streptavidin, neutravidin, an RNA molecule, a small molecule, a nucleic acid molecule, a fluorescent in situ hybridization (FISH) probe, fragment antigen binding region, or nanobody. In some embodiments, the photoreactive compound includes ruthenium-based photocatalyst, iridium-based photocatalyst, Rose Bengal derivatives, fluorescein derivatives, Eosin Y derivatives, Methylene blue derivatives, Flavin derivatives, Lumiflavin, riboflavin, Rhodamine derivatives, Porphyrin derivatives, Quinolinone derivatives, Pterin derivatives, photosensitized protein, miniSOG, Killer Red, phenol, aryl azide, or benzophenone. In some embodiments, the methods may include the step of activating the reactive probe to have a free radical and form a covalent bond with a tyrosine of the biological sample in a selected region of interest. In other words, ruthenium-based compound is delivered to a location of the biological sample. In some embodiments, the method may include the step of illuminating the location of the biological sample to activate the ruthenium-based compound, wherein the activated ruthenium-based compound allows the reactive probe to have the free radical. In some embodiments, the step of illuminating includes illuminating a location for 25 μs/pixel to 400 μs/pixel, for 50 μs/pixel to 300 μs/pixel, or for 75 μs/pixel to 200 μs/pixel. In some embodiments, the reactive group of the reactive probe is configured to generate a reactive intermediate (e.g., phenoxyl radical, carbene or the like) that is responsible for covalent bond formation with amino acid in proximity. In some embodiments, the activated photoreactive probe can promote the reactive probe to have a free radical and form the covalent bond with an amino acid of the biological sample in the selected region of interest. In some embodiments, the amino acid may be alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine or the like.

In some embodiments, the methods may include the step of illuminating the biological sample with an imaging lighting source of an image-guided microscope system. The methods may include the step of imaging the illuminated sample with a camera. The methods may include the step of acquiring with the camera at least one image of subcellular morphology of the sample in a first field of view. The methods may include the step of processing the at least one image and determining a region of interest in the sample based on the processed image. The methods may include the step of obtaining coordinate information of the region of interest. In some embodiments, the methods may include the step of using an alkaline phosphatase to dephosphorylate the biological sample.

In some embodiments, the methods may include the step of delivering an enzyme to a biological sample so as to activate a reactive probe to have a free radical. In certain embodiments, the enzyme includes a peroxidase or a biotin ligase. In some embodiments, the methods may include the step of conjugating a detectable labeling system as shown in FIG. 2D with the reactive probe and detectably proximity labeling neighbors proximal the target molecule (i.e., the prey) with detectable label activity. In some embodiments, the detectable label includes a catalytic label. In some embodiments, the detectably proximity labeling includes photoselective proximity labeling a region less than 5 μm, less than 2 μm, less than 1 μm, less than 500 nm, less than 300 nm, less than 200 nm, or less than 100 nm in diameter. Some embodiments include the step of conjugating the connector 272 with the reactive probe. In some embodiments, the connector is conjugated to the reactive probe through the affinity between the connector and the reactive probe so as to identify the location of the biological sample covalently bound with the reactive probe. Some embodiments further include the step of conjugating a tag-peroxidase to the connector. In some embodiments, the tag-peroxidase is configured to catalyze the subject probe (e.g., tyramide probe) to form a covalent bond between the additional subject probe and the biological sample. In alternative embodiments, the tag-peroxidase is configured to catalyze the reactive probe to form a covalent bond between the reactive probe and the biological sample. More specifically, the tag-peroxidase can activate the additional subject probe to have a free radical and form a covalent bond between the additional subject probe and a tyrosine of the biological sample outside the selectively illumination region. Some embodiments further include the step of removing at least the region of interest from the microscope stage. Some embodiments further include the step of subjecting the selectively illuminated sample to mass spectrometry or sequencing analysis.

In some embodiments, any of the methods herein may include a step of using an affinity matrix to extract the biological sample labelled with the reactive probe, such as when the reactive probe (attached to a molecule) has specific affinity for an affinity matrix. An affinity matrix can a metal affinity matrix or a non-metal affinity matrix. Examples of metal affinity matrix include titanium oxide. A metal affinity matrix can be one or more of TiO₂ bead, TiO₂ column, immobilized metal affinity chromatography (IMAC), and immobilized metal affinity column. In some embodiments, a P portion of a reactive probe forms an ionic bond with a metal affinity matrix so as to pull-down the biological sample labelled with the reactive probe. IMAC typically includes metal ions that have been immobilized by chelation to an insoluble matrix. Molecules (such as phosphates, phosphonates, and other molecules with affinity for metal ions can be separated (enriched or isolated) based on their affinity to the immobilized metal ions.

In some embodiments, this and other methods may include a step of digesting the biological sample with an enzyme such as a protease (e.g., trypsin) so as to generate digested peptides labelled with the reactive probe. In some embodiments, the method may include a step of digesting the biological sample labelled with the reactive probe bound on the metal affinity matrix so as to generate digested peptides labelled with the reactive probe bound on the metal affinity matrix. In some embodiments, the method may include the step of eluting the biological sample labelled with the reactive probe, wherein the biological sample includes a protein. In some embodiments, the methods may include the step of using an affinity bead to extract the biological sample labelled with the reactive probe, wherein the affinity beads interact with the tag of the reactive probe labelling the biological sample. In some embodiments, the methods may further include a step of analyzing the biological sample labelled with the reactive probe through liquid chromatography mass spectrometry.

The methods may include steps of delivering the reactive probe to a biological sample; activating the reactive probe to have a free radical and form a covalent bond with a tyrosine (or another aromatic or sulfur containing amino acid) of the biological sample in a selected region of interest; using a metal affinity matrix to extract the biological sample labelled with the reactive probe; eluting the biological sample labelled with the reactive probe, wherein the biological sample includes a protein; and digesting the biological sample labelled with the reactive probe so as to generate digested peptides labelled with the reactive probe. In this and other embodiments, the protein labelled with reactive probe can be eluted from the metal affinity matrix for digestion in solution instead of on-bead digestion. The digested peptides can be analyzed (immediately or after time) through liquid chromatography mass spectrometry or further processed by extraction by an affinity bead, which interacts with the tag of the reactive probe labelling the biological sample which may significantly reduce the background noise by means of two extraction system including the metal affinity matrix (via P portion of the reactive probe) and the affinity bead (via tag of the reactive probe).

In some embodiments, the methods may include steps of delivering a reactive probe to a biological sample; activating the reactive probe to have a free radical and forming a covalent bond with a tyrosine of the biological sample in a selected region of interest; using a metal affinity matrix to extract the biological sample labelled with the reactive probe; eluting the biological sample labelled with the reactive probe, wherein the biological sample includes a protein; using an affinity bead to extract the biological sample labelled with the reactive probe, wherein the affinity beads interact with the tag of the reactive probe labelling the biological sample; and digesting the biological sample labelled with the reactive probe bound on the affinity bead so as to generate digested peptides labelled with the reactive probe bound on the affinity bead. A protein can be extracted by two systems including the metal affinity matrix and the affinity bead which may (significantly) reduce the background noise, but in some examples, the protein can be digested on the affinity bead in order to increase the sensitivity or signals for protein identification through liquid chromatography mass spectrometry.

Some methods include contacting a biological sample having a target molecule with a photoreactive probe as described herein to non-covalently conjugate a photoreactive probe with the target biomolecule, washing unconjugated photoreactive probe away, using optical radiation to spatially selectively activate the photoreactive probe and thus to induce the bonding between the reactive probe and a nearby molecule in proximity to the target biomolecule, washing unbound reactive probe away, labeling the probe-bearing biomolecule/probe complex with a labeling system, and selectively proximity labeling neighbors proximal the target biomolecule.

The methods may include the step of extracting a plurality of the probe-labelled proteins from the biological sample through an affinity precipitation between the reactive probe and a plurality of affinity beads (e.g., streptavidin magnetic beads). The methods may include the step of subjecting the extracted proteins to mass spectrometry analysis. The methods may include the step of identifying the extracted proteins of the biological sample.

The purification methods for chemical reaction labelling may include the step of delivering a reactive probe to a biological sample; activating the reactive probe to have a free radical and forming a covalent bond with a tyrosine of the biological sample in a selected region of interest; and using an affinity bead to extract the biological sample labelled with the reactive probe, wherein the affinity bead interacts with the tag of the reactive probe labelling the biological sample. Those methods can (significantly) reduce the background noise or non-specific binding by means of the interaction between the affinity bead and the tag of the reactive probe.

FIG. 2F and FIG. 2G illustrate two exemplary methods of pull-down or purification of the biomolecule 211 labelled with the reactive probe 206 (as described elsewhere herein). Although the methods may refer to mass spectrometry analysis by way of example, any of the methods and systems herein can use other methods of analysis, such as sequencing. Referring to FIG. 2F, the first example uses the metal affinity matrix 410 to pull-down the molecule 211, while the metal affinity matrix 410 forms an ionic bond with the P portion 203 of the reactive probe 206. In this example, the metal affinity matrix 410 can be one or more of TiO₂ bead, TiO₂ column, immobilized metal affinity chromatography (IMAC), and immobilized metal affinity column. Other molecules, which cannot interact with P portion 203 of the reactive probe 206, can be washed away so as to obtain the biomolecule 211 with the reactive probe 206 bound on the metal affinity matrix 410. In some embodiments, the biomolecule 211 with the reactive probe 206 bound on the metal affinity matrix 410 can be subject to on-bead digestion and then subject to mass spectrometry analysis. In some embodiments, the biomolecule 211 labelled with the reactive probe 206 can be eluted and digested to form peptides, which can be subject to mass spectrometry analysis. In some embodiments (see FIG. 2G), the eluted biomolecule 211 labelled with the reactive probe 206 can be pulled down by the affinity beads 420, which interact with the tag 201 of the reactive probe 206. The pull-down complex can be subject to the on-bead digestion and then to mass spectrometry (or sequencing or other) analysis. A second example utilizes the affinity bead 420 to purify the biomolecule 211 that has been labelled with the reactive probe 206. Since the affinity bead 420 is able to form an ionic bond with the tag 201 of the reactive probe 206, the affinity bead 420 can be used to purify the biomolecules labelled with the reactive probe 206 while the non-labelled biomolecules can be washed away. Similarly, the affinity bead complex can be used for the on-bead digestion for mass spectrometry analysis or the biomolecule 211 labelled with the reactive probe 206 can be eluted from the complex for trypsin digestion to form peptide 221 labelled with the reactive probe 206. The peptide 221 with the reactive probe 206 could be pulled down with the metal affinity matrix 410 by the previously mentioned ionic bond and the non-interacted peptides can be washed away. After elution, the peptides 221 with the reactive probe 206 can be used in a mass spectrometry analysis.

FIG. 2H and FIG. 2I show two other exemplary methods of pull-down or purification of the biomolecule 211 labelled with the reactive probe 206. In some embodiments shown in FIG. 2E, the biomolecule 211 labelled with the reactive probe 206 is protein. After illumination as previously described, the labelled biomolecule 211 can be digested by enzyme e.g., trypsin or another protease. The digested peptide 221 as shown in FIG. 2H, can be pulled down or purified by the metal affinity matrix 410 or the affinity bead 420. Through the metal affinity matrix 410 pull-down system (refer to FIG. 2H), the digested peptide 221 with reactive probe 206 can be enriched by the interaction with the P portion 203. After enrichment of the digested peptides 221, the digested peptides 221 with the reactive probe 206 can be eluted and analyzed by mass spectrometry. In some embodiments, as illustrated in FIG. 2I, the eluted peptides 221 can be purified or pulled down by the affinity bead 420. Doing so may reduce non-specific binding effect due to the non-specific interaction between either metal affinity matrix 410 or affinity bead 420 and non-specific binding peptides. In an alternative embodiment shown in FIG. 2I, the digested peptide 221 can be pulled down by the affinity bead 420 first and then the metal affinity matrix 410.

### Material and Methods

### Conjugation of Ruthenium-Based Antibody

Ru(bpy)₃ NHS-ester (Bis(2,2′-bipyridine)-4′-methyl-4-carboxy bipyridine-ruthenium N-succinimidyl ester-bis(hexafluorophosphate)) molecules were conjugated to donkey anti-rabbit/donkey anti-mouse IgG antibodies via an amide coupling reaction, as shown in FIG. 9. Three hundred micrograms of antibodies were reacted with Ru(bpy)₃ NHS-ester in a final condition of 0.5 mg/mL antibody and 0.35 mM of Ru(bpy)₃ NHS-ester in 100 mM borate buffer (pH 8.0). The reactions were performed at room temperature for 2 h in the dark. Glycine was added to 100 mM for inactivating the reactions, and the antibody-Ru(bpy)₃ conjugates were then purified by an off-line size exclusion column (PD midiTrap G-25, Cytiva) with a gravity flow. The concentrations of purified antibody-Ru(bpy)₃ conjugates were measured by Pierce™ BCA protein assays (Thermo Fisher Scientific) using the unconjugated antibodies as the standards. For confirming that the Ru(bpy)₃ molecules was conjugated on the antibodies, the purified products were further detected by a NanoDrop spectrophotometer (Thermo Fisher Scientific) at 455 nm wavelength.

### Proposed Reactive Probe Synthesis Scheme

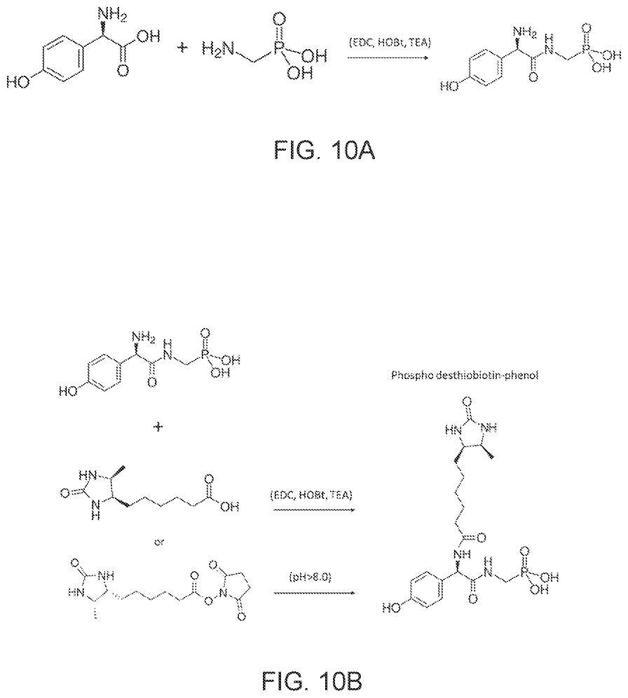

(2R)-2-amino-2-(4-hydroxyphenyl)acetic acid is conjugated with (Aminomethyl)phosphonate to form (2R)-2-amino-2-(4-hydroxyphenyl)acetyl(aminomethyl)phosphonate, as shown in FIG. 10A. (2R)-2-amino-2-(4-hydroxyphenyl)acetyl(aminomethyl)phosphonate is reacted with either desthiobiotin or 1-({6-[(4R,5S)-5-Methyl-2-oxo-4-imidazolidinyl]hexanoyl}oxy)-2,5-pyrrolidinedione to form phosphodesthiobiotin-phenol, as shown in FIG. 10B.

### Cell Preparation

Cells were cultivated at 37 °C in a 5% CO₂ humidified environment in Dulbecco's Modified Eagle Medium supplemented with 10% FBS. 2×10⁵ cells were seeded in glass bottom chambers and incubated for approximately 16 h to 80-90% confluency. Afterwards, cells were washed with PBS and fixed with 2.4% paraformaldehyde (PFA) or methanol. Fixed cells were incubated with PBS/0.5% Triton X-100 to permeabilize the cell membrane, and blocked with 3% BSA in PBS/0.1% Triton X-100 for 1 h, followed by 30 min of 0.002% streptavidin and 15 min of 40 μM biotin blocking.

### Hybridization of Ruthenium-Based Antibody

Cells were incubated with primary antibody for 2 h at room temperature with the following antibodies: rabbit anti-NCL, mouse anti-G3BP1 in blocking buffer (PBS/0.1% Triton X-100 with 3% BSA). After washing with PBS/0.1% Triton X-100, 10 μg/mL of antibody-Ru(bpy)3 conjugates were hybridized to the primary antibody overnight at 4 °C. Cells were subsequently stained with fluorescence marker (goat anti-rabbit AlexaFluor 647 or goat anti-mouse AlexaFluor 647) for 1 h at room temperature.

### Two-Photon Labeling on Subcellular Cell for Quantitative LC-MS/MS Analysis

Cells were incubated with two-photon (2P) labeling reagent containing 5-7 mM desthiobiotin-phenol and 0.005% methyl viologen. Two-photon laser coupled with a microscopic system was used for spatially resolved photolabeling at a laser power of 100-200 mW, and the cells were subjected to a laser-exposure time at 100-1000 microseconds. Labelled cells were washed with the buffer containing 10 mM sodium ascorbate, 5 mM trolox, and 0.02% sodium azide for quenching the photochemical reaction, and cells were washed with PBST three times in total.

### Protein Extraction and On-Bead Digestion

Labelled cells were harvested by scrapping with buffer containing 10 mM Tris (pH 8.0), 1% Triton X-100, 1-fold protease inhibitor cocktail, 10 mM sodium ascorbate, 5 mM Trolox, and 1 mM sodium azide. Harvested cells were sonicated at 60% power using a Q125 sonicator (Qsonica) with 1 s on/2 s off interval for 2 min, then subjected to evaporate the scraping buffer for 2 h by SpeedVac system. 160 μL of lysis buffer containing 4% Sodium dodecyl sulfate (SDS), 1% Triton X-100, 100 mM Tris (pH 8.0), and 20 mM dithiothreitol (DTT) were added to the harvested cells, and the mixture was vortexed at 1 min on/2 min off interval for 5 cycles. To retrieve the cross-linked amide groups resulted from PFA fixation, the lysed cells were further heated at 99 °C for 45 min followed by another vortexing at 1 min on/2 min off interval for 5 cycles. Lysates were centrifuged at 16,000 g for 20 min at 20 °C, and the supernatants were collected. Pierce™ 660 nm Protein Assay (Thermo Fisher Scientific) was used to measure the protein concentrations, and 240 μg of proteins were subjected to the immunoprecipitation. Streptavidin magnetic beads were washed with dilution buffer (0.5% Triton X-100/PBS) three times, and the protein lysates were diluted 10-fold to reduce the SDS concentration to be less than 0.4%, and the diluted lysates were added to the washed beads and incubated at 2-8 °C for 16 h with rotation. After which, the biotin-protein bound beads were washed with the following washing buffers to reduce the non-specific binding: Buffer A (2% SDS, 50 mM Tris (pH 8.0)); Buffer B (0.5M NaCl, 0.1% deoxycholic acid, 0.1% SDS, 1% Triton X-100, 50 mM HEPES); Buffer C (0.5% deoxycholic acid, 0.5% Triton X-100, 10 mM Tris (pH 8.0), 250 mM LiCl). For on-bead digestion, the beads were further washed with 100 μL 50 mM triethylammonium bicarbonate buffer three times, and the biotin-protein bound beads were then mixed with 0.2 μg of Trypsin/Lys-C (V5071, Promega) in a final volume of 20 μL at 37 °C for 100 min for an initial digestion. After that, the supernatants were collected and subjected to the overnight digestion without adding further enzyme. Finally, the digests were acidified by adding 2 μL of 10% formic acid, and were desalted by C18 Ziptip. Desalted peptides were dried by Speedvac and stored at −20 °C prior to LC-MS/MS analysis.

### LC-MS/MS Analysis

### Detection of Immunoprecipitated Product by Data-Dependent Acquisition Mass Spectrometry

LC-MS/MS analysis was performed using an UltiMate 3000 RSLCnano system (Thermo Fisher Scientific) coupled to an Orbitrap Fusion Lumos mass spectrometer (Thermo Fisher Scientific). The desalted peptides were resuspended in 0.1% formic acid in water and loaded onto a PepMap™ 100 C18 HPLC column (2 μm, 100 angstrom, 75 μm×25 cm; Thermo Fisher Scientific), and peptides were eluted over 160 min gradients for nuclei-illuminated samples, over 120 min gradients for nucleoli-, SG-illuminated samples. The full MS spectra ranging from m/z 375-1500 were acquired at a resolving power of 120,000 in Orbitrap, an AGC target value of 4×10⁵, and a maximum injection time of 50 ms. Fragment ion spectra were recorded in the top-speed mode at a resolving power of 30,000 in Orbitrap using a data-dependent method. Monoisotopic precursor ions were selected by the quadrupole using an isolation window of 1.2, 0.7, 0.4 Th for the ion with 2+, 3+, 4-7 charge states, respectively. An AGC target of 5×10⁴, maximum injection time of 54 ms, higher-energy collisional dissociation (HCD) fragmentation with 30% collision energy, and a maximum cycle time of 3 s were all applied. Dynamic exclusion was set to 60 s with an exclusion window of 10 ppm. Precursor ions with the charge state of unassigned, 1+, or superior to 8+ were excluded from fragmentation selection.

### Protein Identification and Label-Free Quantification

Raw data from the same batch of two-photon illumination were processed together with Proteome Discoverer (Thermo Fisher Scientific) by Sequest HT algorithm against the UniProtKB/Swiss-Prot human protein database (version 2020.02, 20,365 entries) for feature extraction, peptide identification, and protein inference. Database search was performed as follows: tryptic peptides with up to three missed cleavages; mass tolerances of 10 ppm for peptide ions, and 0.05 Da for fragment ions; static carbamidomethylation (+57.0215 Da) on Cys residues; dynamic deamidation (+0.9840 Da) on Asp and Gln residues, oxidation (15.9949 Da) on Met residues, acetylation on protein N-termini (+42.0106 Da), and desthiobiotin phenol modification (+331.1896) on Tyr residues. The minimal peptide length was set as 6 residues. The false discovery rate (FDR) of peptide and protein were both set as 1%.

For label-free quantification, the time window for chromatographic peak alignment was set at 20 min. Peptide level data was then normalized to the total peptide intensity, and the quantification value for a given protein was derived from the sum of normalized intensities of the top three intense unique peptides belonging to that protein.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

1. A reactive probe comprising a formula (I): 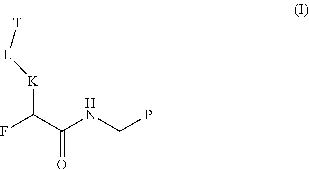
wherein
P is either a phosphate group or a phosphonate group;
K is a connecting group including —(CH₂)ₘ—Y—, wherein m is an integer ranging from 0 to 6, and Y is selected from the group consisting of 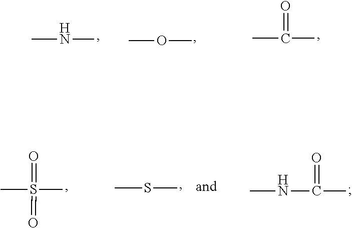
L is a linker between K and T;
F is a reactive group; and
T is a tag moiety.

2. The reactive probe of claim 1, wherein the linker L has fewer than 24 atoms in a continuous chain or is a covalent bond.

3. (canceled)

4. The reactive probe of claim 1, wherein the linker L is 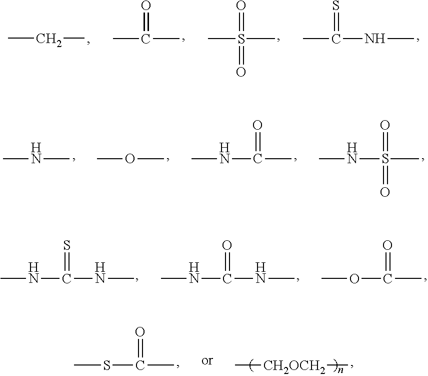
wherein n is an integer ranging from 1 to 6.

5. (canceled)

6. The reactive probe of claim 1, wherein m is selected from 0, 1, 2, and 3, and Y is

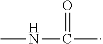

7. The reactive probe of claim 1, wherein the tag moiety is a click chemistry moiety selected from the group consisting of an alkyne-based moiety and an azide-based moiety.

8. The reactive probe of claim 7, wherein the click chemistry moiety comprises the moiety of

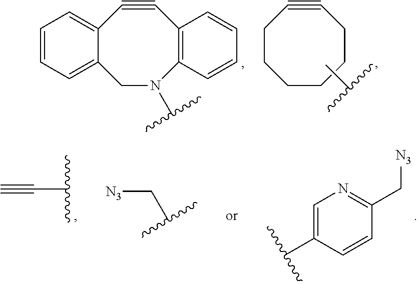

9. The reactive probe of claim 1, wherein the tag moiety is a biotin derivative selected from the group consisting of

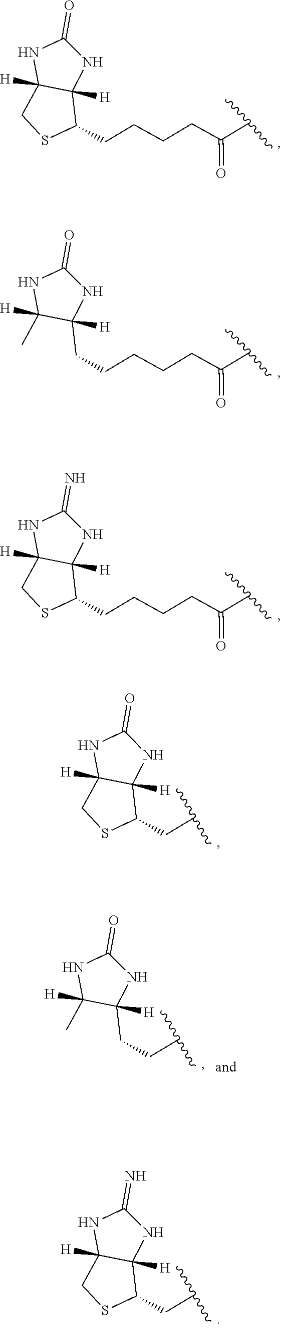

10. The reactive probe of claim 1, wherein the reactive group comprises a phenolic group, an aryl azide moiety, a benzophenone, a phenoxyl radical trapper, a nucleobase-specific 3-cyanovinylcarbazole nucleoside (CNVK), or a diazirine.

11. The reactive probe of claim 10, wherein the reactive group is the phenolic group, and the phenolic group comprises the moiety of

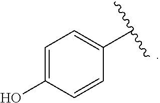 [SMILES: *c1ccc(O)cc1]

12.-17. (canceled)

18. The reactive probe of claim 1, wherein the reactive probe is selected from the group consisting of

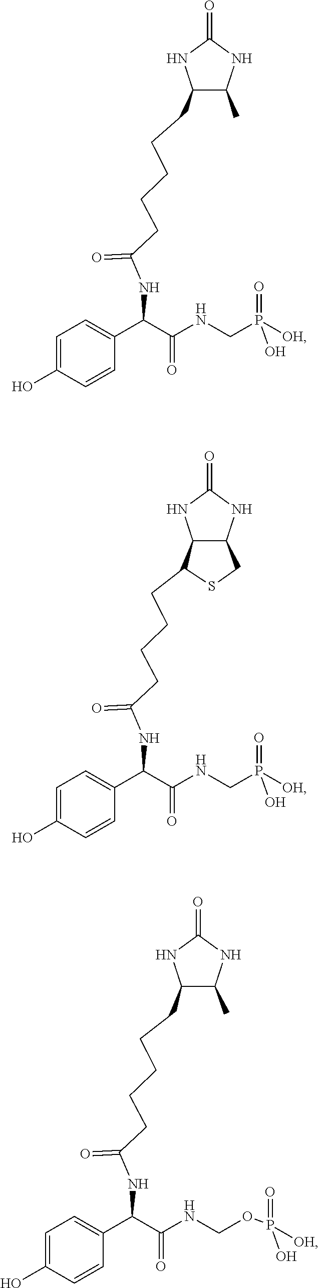

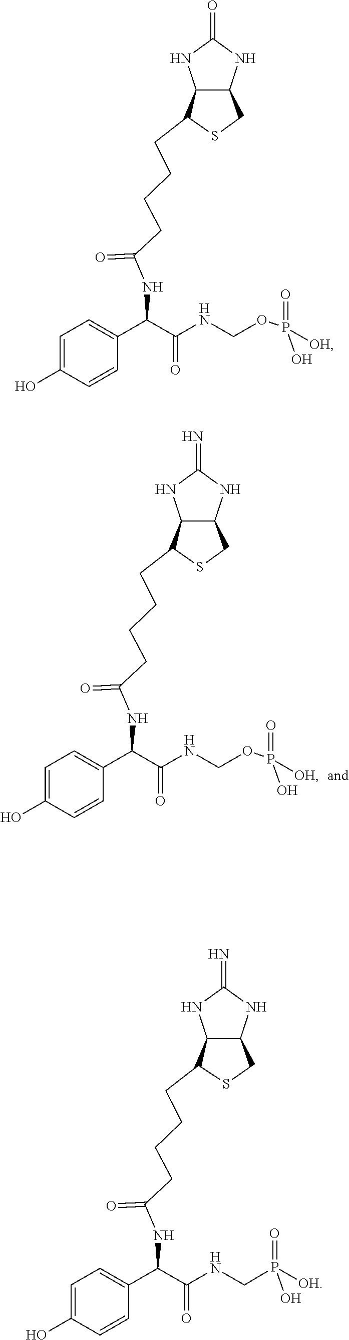

19. A purification and pull-down method for chemical reaction labelling, comprising:
(a) delivering the reactive probe as claimed in claim 1 to a biological sample;
(b) activating, in a selected region of interest in the biological sample, the reactive probe to have a free radical to thereby generate an activated reactive probe;
(c) forming, based on the free radical, a covalent bond between the activated reactive probe and a biomolecule of interest in the biological sample;
(d) contacting the biological sample with a first affinity matrix to thereby extract the biomolecule of interest labelled with the reactive probe;
(e) eluting, from the first affinity matrix, the biological sample labelled with the reactive probe;
(f) contacting the biomolecule of interest labelled with the reactive probe of step (e) with a second affinity matrix to thereby purify the biomolecule of interest labelled with the reactive probe; and
(g) eluting, from the second affinity matrix, the biomolecule of interest labelled with the reactive probe.

20.-21. (canceled)

22. The purification and pull-down method of claim 19, further comprising steps of delivering a photoreactive compound to the biological sample; and illuminating the selected region of interest of the biological sample to activate the photoreactive compound therein, wherein the activated photoreactive compound causes the reactive probe to have the free radical.

23. The purification and pull-down method of claim 19, wherein the first affinity matrix is a metal affinity matrix, and the second affinity matrix includes affinity beads which interact with the tag of the reactive probe labelling the biological sample.

24. The purification and pull-down method of claim 19, wherein the first affinity matrix includes affinity beads which interact with the tag of the reactive probe labelling the biological sample, and the second affinity matrix is a metal affinity matrix.

25. The purification and pull-down method of claim 24, wherein the metal affinity matrix is one or more of TiO₂ beads, a TiO₂ column, immobilized metal affinity chromatography (IMAC) beads, or an immobilized metal affinity column.

26. (canceled)

27. The purification and pull-down method of claim 19, further comprising a step of using an alkaline phosphatase to dephosphorylate the biological sample.

28. The purification and pull-down method of claim 19, further comprising a step of digesting the biomolecule of interest labelled with the reactive probe bound to the first affinity matrix or the second affinity matrix with a protease so as to generate digested peptides labelled with the reactive probe bound on the first affinity matrix or the second affinity matrix.

29.-30. (canceled)

31. The purification and pull-down method of claim 28, further comprising a step of analyzing the digested peptides labelled with the reactive probe through liquid chromatography mass spectrometry.

32. (canceled)

33. The purification and pull-down method of claim 23, wherein the metal affinity matrix is one or more of TiO₂ beads, a TiO₂ column, immobilized metal affinity chromatography (IMAC) beads, or an immobilized metal affinity column.
